(19) United States
(12) Patent Application Publication
(10) Pub. No.: US 2004/0009911 A1
(43) Pub. Date: Jan. 15, 2004

(54) Hepsin substrates and prodrugs

(75) Inventors: Jennifer Leslie Harris, San Diego, CA (US); Bradley J. Backes, Chicago, IL (US); Jun Li, San Diego, CA (US); David C. Tully, San Diego, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (BM)

(21) Appl. No.: 10/356,656

(22) Filed: Jan. 31, 2003

Related U.S. Application Data
(60) Provisional application No. 60/421,109, filed on Jan. 31, 2002.

(51) Int. Cl. A61K 38/10; A61K 38/08; C07K 7/08; C07K 7/06

(52) U.S. Cl. 514/12; 514/13; 514/14; 514/15; 514/16; 514/17; 530/324; 530/325; 530/326; 530/327; 530/328; 530/329; 530/330

(57) ABSTRACT

Substrate specificity profiles are used to determine optimal hepsin substrate sequences, both to the prime side and non-prime side of the hepsin recognition site. The hepsin substrate sequences are used in designing substrates, inhibitors, and prodrugs. For example, prodrugs are provided for use in the treatment of prostate cancer. Hepsin inhibitors based on substrate specificity are also provided.

### CROSS-REFERENCE TO RELATED APPLICATIONS

This application is related to U.S. nonprovisional application U.S. Ser. No. 10/066,541 filed Jan. 31, 2002, and converted to provisional application U.S. S No. 60/421,109 on Nov. 27, 2002, titled “Hepsin Substrates and ProDrugs.” The present application claims priority to, and benefit of, this application, pursuant to 35 U.S.C. §119(e) and any other applicable statute or rule.

### COPYRIGHT NOTIFICATION

Pursuant to 37 C.F.R. 1.71(e), a portion of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to substrate specificity and protein substrate design. More particularly, the present invention relates to substrate design for targeting and/or inhibition of hepsin enzyme activity.

### BACKGROUND OF THE INVENTION

Substrate specificity of an enzyme is an important characteristic that governs its biological activity. Characterization of substrate specificity provides invaluable information useful for a complete understanding of often complex biological pathways. In addition, substrate specificity profiles are useful in the design of selective substrates, inhibitors, and prodrugs directed to enzymatic targets.

Proteases, also known as proteinases, peptidases, or proteolytic enzymes, are enzymes that degrade proteins by hydrolyzing peptide bonds between amino acid residues. Various categories of proteases include thiol proteases, acid proteases, serine proteases, metalloproteases, cysteine proteases, carboxyl proteases, and the like.

Hepsin is a member of an important family of enzymes, the membrane associated serine proteases. Hepsin is a 51 kDa protein comprising 417 amino acids that was originally isolated from cDNA clones isolated from human liver cDNA libraries. The protease typically contains a short hydrophobic amino acid sequence in the region near the amino terminus, and the carboxyl terminus is similar to a typical serine protease. It is primarily located within the plasma membrane of cells with the C-terminus positioned at the external surface of the cells. See, e.g., Tsuji (1991) J. Biol. Chem. 266:16948-16953. Hepsin is thought to play a role in cell growth and is known to be produced at a particularly high level in the liver as well as in human hepatoma cells, some cancer cells and nerve cells. See, e.g., Torres-Rosado (1993) Proc. Natl. Acad. Sci. USA 90:7181-7185.

Many proteases are non-specific in their activity, e.g., they digest proteins to peptides and/or amino acids. Other proteases are more specific, e.g., cleaving only a particular protein or only between certain predetermined amino acids. Still other proteases have optimal sequences that they cleave preferentially over others. The substrate specificity of hepsin has not been determined, and its availability as a prodrug target has not been previously explored. Improved methods of identifying the optimal substrates of proteases, such as hepsin, are desirable. The present invention fulfills these needs, as well as other needs that will be apparent upon complete review of this disclosure.

### SUMMARY OF THE INVENTION

The present invention provides hepsin substrates, prodrugs, diagnostics and inhibitors, as well as methods involving the hepsin substrates and vectors useful in expressing hepsin. Hepsin is a membrane-associated serine protease which is upregulated in some disease states, e.g., in prostate tumors, making hepsin a potential drug target. The present invention provides a substrate specificity profile for hepsin, in addition to identifying prodrugs useful for treating cancers, e.g., prostate cancer. The hepsin-targeted prodrugs of the present invention comprise a hepsin-cleavable molecule, which typically includes a hepsin recognition site or substrate and a therapeutic, diagnostic, or cell modulating moiety that is released when the molecule is cleaved by hepsin. Therefore, a tumor site that has a high level of hepsin can be directly targeted by the prodrugs of the present invention. Preferably, the prodrug produces an effect after cleavage by the hepsin protease (e.g., after release and/or activation of the drug), and is therefore not active in areas that do not contain hepsin. This results in the prodrugs being significantly less toxic that many cancer therapeutics.

In one aspect, the present invention provides hepsin-cleavable molecules having a hepsin cleavage site. The hepsin-cleavable molecules of the invention typically comprise:

P₄P₃P₂P₁X

wherein P₁-P₄ each comprise an amino acid or amino acid-like moiety, and X comprises an additional substrate moiety (e.g. a prime-side moiety). For example, P₁ is typically arginine or lysine; P₂ is typically valine, leucine, isoleucine, methionine, norleucine, arginine, histidine, lysine, asparagine, or threonine; P₃ is typically arginine, lysine, histidine, glutamine, serine, or threonine; and P₄ is typically arginine, lysine, proline, valine, leucine, isoleucine, methionine, norleucine, alanine, glycine, tryptophan, phenylalanine, or tyrosine, preferably arginine, lysine, proline, valine, leucine, or alanine. Optionally, the amino group of the N-terminal amino acid (e.g., P₄) is derivatized or blocked (e.g., an N-acetylated amino acid). X typically comprises one or more cell modulating moieties, label moieties, or a polypeptide (e.g., a polypeptide comprising about 1 to about 25 amino acids, e.g., a tetrapeptide, or a polypeptide that is not attached to P₄P₃P₂P₁ in a naturally occurring protein, e.g., a non-native peptide sequence). The hepsin cleavage site is between P₁ and X. Exemplary amino acid sequences for P₄P₃P₂P₁ include, but are not limited to, KRLR, KQLR, PQLR, RQLR, RRLR, PRLR, PKLK, PKLR and PRLK.

In some embodiments of the present invention, X comprises a prime side amino acid sequence as follows:

P₁′P₂′P₃′P₄′

wherein P₁′ is typically methionine, norleucine, leucine, isoleucine, valine, alanine, tyrosine, or threonine; P₂′ is typically alanine, phenylalanine, tyrosine, threonine, histidine, P₃′ is typically arginine, lysine, histidine, glutamine, serine, threonine, tyrosine, tryptophan, glycine, leucine or methionine; and P₄′ is aspartic acid, glycine, proline, valine, or methionine. The prime side sequence forms one side (the C-terminal side) of the cleavage site while the non-prime sequence forms the other (N-terminal) side. Optionally, the C-terminal carboxyl group of the prime-side sequence is derivatized or blocked (e.g., by C-terminal amidation, or the presence of an alcohol, methyl amide, or ethyl amide moiety).

In other embodiments of the present invention, X comprises a cell modulating moiety such as a cytotoxic moiety, an antiproliferative moiety, an anti-metastatic moiety, an apoptosis-inducing moiety, a necrosis-inducing moiety, or the like. Cytotoxic moieties include, but are not limited to, bacterial toxins, doxorubicin, daunorubicin, epirubicin, idarubicin, anthracycline, paclitaxel, camptothesin, mitomycin C, phenylenediamine mustard, and the like. Typically, a cell modulating moiety is inactive until cleaved from the hepsin-cleavable molecule, e.g., by hepsin.

In other embodiments, X comprises a label moiety, e.g., for diagnostic uses. Label moieties of the invention include, but are not limited to, absorbent, fluorescent, or luminescent label moieties. Exemplary label moieties include, but are not limited to fluorophores, coumarin moieties, such as 7-amino-4-carbamoylcoumarin, 7-amino-3-carbamoyl-4-methylcoumarin, or 7-amino-4-methylcoumarin, or rhodamine moieties. Typically, a label moiety exhibits significantly less absorbance, fluorescence or luminescence when attached to the hepsin-cleavable molecule than when released from the hepsin-cleavable molecule. The label moiety an be attached directly to the P₁ substituent, or alternatively it can be attached to the P₁ substituent via a linker or spacer molecule (e.g., a tetrapeptide or a self-immolative linker).

In some embodiments, the hepsin-cleavable molecule comprises a fluorescence resonance transfer energy pair. A first member of the pair is typically attached to the molecule on one side of the hepsin cleavage site and a second member is attached to the molecule on the opposite side of the hepsin cleavage site. Exemplary pairs include, but are not limited to: amino benzoic acid and nitro-tyrosine; 7-methoxy-3-carbamoyl-4-methylcoumarin and dinitrophenol; and, 7-dimethylamino-3-carbamoyl-4-methylcoumarin and dabsyl.

In other embodiments, the label moiety comprises a first quantum dot attached to the molecule on one side of the hepsin cleavage site and a second quantum dot attached to the molecule on the opposite side of the hepsin cleavage site. Typically, the first and second quantum dots emit signals of different wavelengths upon illumination.

In another aspect, the present invention provides anti-cancer prodrugs, such as a prodrug directed to prostate cancer. The prodrugs typically comprise a peptide sequence, e.g., a non-prime side sequence, and a cytotoxic moiety as described above. The cytotoxic moiety is typically attached to the peptide sequence, e.g., at P₁, and is inactive until the peptide sequence is cleaved by hepsin. The peptide sequences of the prodrugs, likewise optionally comprise an additional peptides sequence, e.g., a prime side sequence, as defined above.

In another aspect, the present invention provides hepsin-cleavable peptides comprising fewer than 25 amino acids, and having the core structure:

P₄P₃P₂P₁

and having one or more amino acids attached to either or both of P₁ and P₄, wherein P₁-P₄ are defined as described above. For example, in one embodiment P₁ is arginine, P₂ is leucine, P₃ is arginine or asparagine, and P₄ is lysine or proline, wherein the sequence further comprises 1 to 20 amino acids linked to P₄ or P₁. In some embodiments, the additional amino acids include a prime side sequence (e.g., P₁′P₂′P₃′P₄′) as described above,

In other embodiments, the hepsin cleavable peptides of the invention comprise P₁-P₄ as described above and one or more molecules, e.g., peptides, carbohydrates, polyalcohols such as polyethylene glycol, biotin, or crosslinking agents, attached to either or both of P₁ and P₄. Preferably, the molecules attached to the peptide sequence are not typically found attached to native or naturally occurring protein sequences comprising P₁-P₄. In addition to the one or more molecules, e.g., non native molecules, the hepsin cleavable peptides optionally comprise a prime side sequence as described above, e.g., P₁′P₂′P₃′P₄′.

In another aspect, the present invention provides libraries of putative hepsin substrates. Each member of the library typically comprises a putative hepsin cleavage site, which comprises one or more non-prime positions and one or more prime positions. The prime positions and the non-prime positions flank the putative hepsin cleavage site. The one or more non-prime positions are typically occupied by one or more preselected amino acids or amino acid mimetics, which are preselected to allow cleavage of the putative substrate at the putative cleavage site. For example, the preselected non-prime positions optionally comprise P₁-P₄, as described above. The one or more prime positions are also typically occupied by one or more amino acids or amino acid mimetics. The amino acids or substrate moieties in the prime positions typically vary among the members of the library of putative hepsin substrates. For example, those described above, e.g., P₁′P₂′P₃′P₄′, are optionally included in the library. However, the positions can vary to include all known amino acids and/or amino acid mimetics. Furthermore, prime-side substrate peptide sequences absent the non-prime side sequence such as those described above (e.g., P₁′P₂′P₃′P₄′), are optionally also included in the library.

In some embodiments, the putative hepsin substrates further comprise a fluorescence resonance energy transfer pair. The first member is typically coupled to one or more prime position and the second member is typically coupled to one or more non-prime positions. Exemplary pairs are described above.

In another aspect, the present invention provides diagnostic compounds, e.g., fluorescently labeled diagnostic compounds that are used to screen for the presence of hepsin. The diagnostic compounds of the invention typically comprise an amino acid sequence as described above (e.g., KRLR, KQLR, PQLR, RQLR, RRLR, PRLR, PKLK, PKLR, PRLK, and the like) and a label moiety. Label moieties of the invention typically comprise a chromaphore, a fluorophore, a coumarin moiety, a rhodamine moiety, a fluorescence resonance transfer energy pair, or the like. Exemplary coumarin moieties and fluorescence transfer pairs are provided above.

In another aspect, the present invention provides hepsin inhibitors. The inhibitors typically comprise a hepsin recognition site such as a peptide sequence as described above and are linked to an inhibitory moiety, Z. For example, a typical inhibitor is shown below:

P₄P₃P₂P₁Z

wherein P₁-P₄ are defined as described above and Z comprises a transition state analog, a mechanism-based inhibitor, an electron withdrawing group, or the like. Contacting hepsin with the hepsin inhibitors of the invention results in complete or partial inactivation of hepsin. In some inhibitor embodiments, P₄ comprises acetyl lysine. The transition state analog, mechanism-based moiety, or electron withdrawing moiety optionally comprises a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide, or the like. An exemplary hepsin inhibitor of the present invention is a compound having formula I:

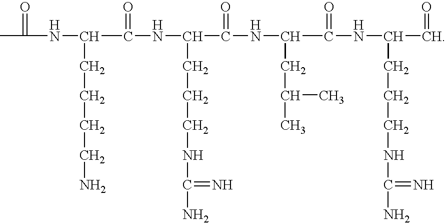

The methods of the present invention also include methods of obtaining a substrate profile for a hepsin activity. The method include the steps of (a) providing a library of putative hepsin substrates, each of which comprises a putative hepsin recognition site, (b) incubating the library in the presence of the hepsin; and (c) monitoring cleavage of the putative hepsin substrates by the hepsin, thereby providing the substrate profile for the hepsin. Preferably, the putative hepsin recognition site comprises one or more non-prime positions and one or more prime positions, each of which positions is occupied by a substrate moiety, wherein the prime and non-prime positions flank a putative hepsin cleavage site; in this embodiment, the substrate moieties that occupy one or more of the non-prime positions are preselected to allow cleavage of the substrate at the putative hepsin cleavage site by the hepsin; and the substrate moieties that occupy one or more of the prime positions vary among different members of the library of hepsin substrates.

Optionally, the putative hepsin substrates further comprise a fluorescence resonance energy transfer pair. In one embodiment, a fluorescence donor moiety and a fluorescence acceptor moiety are attached to the putative hepsin substrates on opposite sides of the putative hepsin cleavage site, such that monitoring the cleavage of the putative hepsin substrates is performed by detecting a fluorescence resonance energy transfer. Monitoring can include detecting a shift in the excitation and/or emission maxima of the fluorescence acceptor moiety, which shift results from release of the fluorescence acceptor moiety from the putative hepsin substrate by the hepsin activity.

Optionally, the one or more non-prime positions of the putative hepsin substrates employed in the methods include a tetrapeptide sequence. Exemplary tetrapeptide sequences include, but are not limited to, KRLR, KQLR, PQLR, RQLR, RRLR, PRLR, PKLK, PKLR and PRLK.

In another aspect, the present invention provides methods of screening a library of compounds for a modulator of hepsin activity. The screening methods include the steps of (a) providing a first library comprising a plurality of putative hepsin substrates having a structure P₄P₃P₂P₁X, wherein P₁, P₂, P₃ and P₄ comprise substrate moieties at non-prime positions and X comprises a label moiety; (b) analyzing the first library to identify substrate moieties at one or more non-prime positions that result in cleavage of the putative hepsin substrate between P₁ and X by a hepsin protease; (c) constructing a second library comprising the identified substrate moieties; (d) incubating the second library with the hepsin protease; and (e) monitoring fluorescence resonance energy transfer between the members of the FRET pair, to identify one or more optimal prime substrate moieties, thereby screening a library of compounds for a modulator of hepsin activity and providing the substrate profile for the enzyme. Constructing the second library typically involves (i) coupling a first member of a fluorescence resonance energy transfer (FRET) pair to a substrate moiety on an N-terminal side of a putative hepsin cleavage site, wherein the substrate moiety comprises an identified substrate moiety from the first library; (ii) coupling a second member of the FRET pair to a substrate moiety on a C-terminal side of the putative hepsin cleavage site; and (iii) linking the compounds of (i) and (ii) together to form members of the second library.

Optional FRET pairs for use in the screening methods include, but are not limited to, amino benzoic acid and nitro-tyrosine; 7-methoxy-4-carbomoylmethylcoumarin and dinitrophenol-lysine, or 7-dimethylamino-4-carbomoylmethylcoumarin and Dabsyl-Lysine. Furthermore, the substrate moiety X on the C-terminal side of the hepsin cleavage site (i.e., the prime side substrate moieties) optionally comprises a tetrapeptide, such as P₁′P₂′P₃′P₄′, wherein P₁′ is attached to P₁ and is methionine, norleucine, leucine, isoleucine, valine, alanine, tyrosine, or threonine; P₂′ is alanine, phenylalanine, tyrosine, threonine, or histidine; P₃′ is arginine, lysine, histidine, glutamine, serine, threonine, tyrosine, tryptophan, glycine, leucine or methionine; and P₄′ is attached to the label moiety and is aspartic acid, glycine, proline, valine, or methionine.

The compositions of the present invention can also be used in methods of inhibiting a hepsin activity, methods of labeling a cell, and/or methods of killing a cell. The methods involve contacting a cell with the appropriate hepsin substrate molecule of the present invention (e.g., a hepsin substrate coupled to an inhibitor moiety, a label moiety, or a cell modulatory or cytotoxic moiety). Optionally, the methods are performed either in vitro or in vivo.

In one aspect, the present invention provides methods of reducing a hepsin activity in a cell. The methods involve contacting the cell with a hepsin inhibitor molecule containing a hepsin recognition site. Typically, the hepsin inhibitor molecule comprises a compound comprising the structure P₄P₃P₂P₁X, wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and wherein X comprises an inhibitory moiety, such as a transition state analog, a mechanism-based inhibitor, or an electron withdrawing group. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide.

As another aspect, the present method provides methods of killing a cell, the methods comprising contacting the cell with a hepsin-cleavable molecule that comprises a hepsin cleavage site, wherein the hepsin-cleavable molecule comprises P₄P₃P₂P₁X, wherein the hepsin cleavage site is between P₁ and X; and wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a cytotoxic moiety. Exemplary cytotoxic moieties for use in the methods of the present invention include, but are not limited to, doxorubicin, daunorubicin, epirubicin, idarubicin, anthracycline, paclitaxel, camptothecin, mitomycin C, phenylenediamine mustard, one or more bacterial toxins, or a combination thereof. Optionally, the cell to be killed comprises a mammalian cell, such as a human cell, a cancer cell, or a cell overexpressing a hepsin activity. In one embodiment of the methods, contacting the cell with a hepsin-cleavable molecule is performed in vitro, such as performing an in vitro assay. In an alternate embodiment, contacting the cell comprises administering the hepsin cleavable molecule to the cell in vivo.

Furthermore, the present method provides methods of screening an individual for increased hepsin activity or expression, using the compositions of the present invention. The methods include the steps of a) obtaining a cell or tissue sample from the individual; b) contacting the cell or tissue sample with one or more hepsin-cleavable molecules that comprise a hepsin cleavage site (for example, a hepsin substrate molecule P₄P₃P₂P₁X wherein the hepsin cleavage site is between P₁ and X; and wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and wherein X comprises a label moiety); and c) detecting a release of the label moiety from the hepsin cleavable molecule, thereby screening the individual for increased hepsin activity or expression. Optionally, the level of detected label is compared to a control or standard level of hepsin activity, thereby determining whether the hepsin activity or expression is increased. These methods performed on a cell from prostate tissue can be used as an indication of prostate cancer.

In yet another aspect, the present invention provides expression vectors for expression of hepsin polypeptides, e.g., in insect cells. The expression vectors typically comprise the following operably linked components: a promoter that is active in the selected cell type (e.g., insect cells); a polynucleotide that encodes a secretion signal polypeptide; and a polynucleotide that encodes the hepsin polypeptide of interest. The hepsin polypeptides typically comprises a hepsin catalytic domain and prodomain, and optionally lack a transmembrane domain. A typical secretion signal polypeptide is a non-hepsin secretion signal polypeptide, such as a honeybee melittin secretion signal polypeptide. In addition, the expression vectors optionally further comprise a polynucleotide that encodes a tag, such as a polyhistidine tag, to facilitate purification of the hepsin polypeptide.

### BRIEF DESCRIPTION OF THE FIGURES

FIG. 1 provides an exemplary P₄P₃P₂P₁X molecule for use in positional scanning techniques to determine non-prime side substrate specificity.

FIGS. 2A, 2B and 2C depicts exemplary prodrugs of the present invention. FIG. 2A depicts a prodrug comprising the amino acid sequence PRLR linked to doxorubicin. FIG. 2B depicts a prodrug comprising the amino acid sequence PKLK linked to camptothecin. FIG. 2C depicts a prodrug comprising the amino acid sequence PKLK linked to doxorubicin.

FIG. 3 provides data illustrating hepsin substrate specificity for arginine in the P₁ position.

FIG. 4, panels A through E further illustrate the substrate specificity of hepsin, as depicted in both 2-dimensional and 3-dimensional graphs. Panel A provides substrate specificity for the P₂ position, Panel B for the P₃ position, and Panel C for the P₄ position. Panel D is an expansion of the specificity data for substrates in which P₁=Arg, and Panel E is an expansion of the specificity data for substrates in which P₁=Lys.

FIG. 5 provides a hepsin amino acid sequence. The prodomain and the catalytic domain (amino acids 47-417) are shown in bold, with the catalytic domain (amino acids 163-417) underlined.

FIG. 6 depicts an exemplary expression vector of the present invention.

FIG. 7, panels A, B, and C depict substrate specificity profiles for hepsin obtained using a baculovirus expression system. Panel A provides substrate specificity for the P₂ position, Panel B for the P₃ position, and Panel C for the P₄ position.

FIG. 8, panels A, B, and C depict substrate specificity profiles for polyhistidine tagged hepsin obtained using a baculovirus expression system. Panel A provides substrate specificity for the P₂ position, Panel B for the P₃ position, and Panel C for the P₄ position.

FIGS. 9A through 9D depict prime-side substrate specificity profiles for hepsin. FIG. 9A provides substrate specificity for the P₁′ position, FIG. 9B for the P₂′ position, FIG. 9C for the P₃′ position, and FIG. 9D for the P₄′ position.

FIG. 10 is a graph illustrating the activity of hepsin toward pro-urokinase plasminogen activator as a substrate.

FIG. 11 provides an exemplary library member for a library of putative hepsin substrates for determining prime side specificity, including a variety of donor and acceptor moieties for use as label moieties (e.g., donor acceptor fluorescence resonance energy transfer pairs).

### DETAILED DESCRIPTION

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a hepsin substrate” includes a combination of two or more substrates; reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

The present invention provides hepsin substrates, prodrugs, diagnostics, and hepsin inhibitors as well as libraries of putative hepsin substrates and expression vectors for producing active hepsin molecules. Hepsin is a cell surface serine protease involved with mammalian cell growth, and has been found to be associated with prostate cancer. Therefore, the inhibitors, substrates, prodrugs, and diagnostic compounds of the present invention are useful in treating and diagnosing prostate cancer (or other cancers with which this protease is associated).

In some embodiments of the present invention, active hepsin is optionally expressed in an E. coli, baculovirus, or other available expression system and can be used to generate a substrate specificity profile, e.g., a profile comprising primary and extended specificity on one or both sides of the cleavage site of hepsin, e.g., the prime and/or non-prime sides of the cleavage site. For example, positional scanning formats are optionally used with tetrapeptide libraries of putative substrates to provide a substrate profile. Substrates are identified, synthesized and tested for hepsin cleavage. In addition, the substrate profile is optionally used to develop hepsin inhibitors and prodrugs, e.g., compositions that can be selectively activated (e.g., cleaved and released) at the cancer site. Furthermore, the specificity information can optionally be used to identify physiological substrates and biological pathways in which hepsin operates.

In one embodiment of the present invention, the substrate specificity information obtained for hepsin is optionally used to design sequences into small molecule substrates using fluorescence resonance energy transfer or other fluorescent or chromagenic signals to observe hepsin activity in vitro, ex vivo, or in vivo. In another embodiment, the sequences are optionally designed into a prodrug format in which the drug is only activated and/or released at sites where hepsin is expressed, e.g., at a cancer site. The selective sequence information can also optionally be used to design fusion proteins or peptides, that when cleaved by hepsin, release a cytotoxic substance, an apoptosis or necrosis inducing signal, or an anti-metastasis signal. Furthermore, the compositions of the present invention are useful in identifying macromolecules as potential downstream substrates of hepsin, thereby enabling identification of the biological pathways through which hepsin acts.

The following discussion details novel hepsin substrates and libraries of putative hepsin substrates, e.g., for screening applications, as well as hepsin expression systems. In addition, the invention provides novel prodrugs (e.g., for treatment of prostate cancer), diagnostic tools, and hepsin inhibitors based on the identified hepsin substrates provided herein. Examples are also provided.

Hepsin and Hepsin Substrates

A typical enzyme of interest in the present invention is hepsin, a serine protease. “Protease,” as used herein, typically refers to an enzyme that degrades proteins or peptides, e.g., by hydrolyzing peptide bonds between amino acid residues. In the present invention, peptides and peptide-like substrates (mimetics) that are cleavable by hepsin are provided.

The terms “polypeptide,” “peptide,” and “protein” are used interchangeably herein to refer to a polymer of amino acids linked through peptide bonds. Polypeptides of the invention include, but are not limited to, proteins, biotinylated proteins, isolated proteins, recombinant proteins, enzymes, and enzyme substrates. In addition, the polypeptides or proteins of the invention optionally include naturally occurring amino acids as well as amino acid analogs and/or mimetics of naturally occurring amino acids, e.g., that function in a manner similar to naturally occurring amino acids. In the present invention, peptides are also optionally constructed using amino acids analogs, derivatives, isomers (e.g., L or D forms of the amino acids), and/or conservative substitutions of the sequences provided herein.

As used herein, a conservative substitution refers to the replacement of one amino acid with a chemically-similar residue, e.g., the substitution of one hydrophobic residue for another. Exemplary substitutions include, but are not limited to, substituting alanine, threonine, and serine for each other, asparagine for glutamine, arginine for lysine, and the like. For example, the present invention provides various tetrapeptide substrate sequences that are cleavable by hepsin. Peptides comprising one or more conservative substitutions of these sequences which are also cleavable by hepsin provide alternate embodiments of the invention. Furthermore, the peptides provided optionally comprise fewer than or greater than four amino acids, e.g., when the remaining amino acids still provide a hepsin cleavable sequence.

Hepsin, a membrane associated serine protease, is a 51 kDa protein comprising 417 amino acids, which was originally isolated from cDNA clones isolated from human liver cDNA libraries. It contains a short hydrophobic amino acid sequence in the region near the amino terminus and the carboxyl terminus is similar to a typical serine protease. The amino-terminus is primarily located within the plasma membrane of cells with the C-terminus at the external surface of the cells. See, e.g., Tsuji et al. (1991) J. Biol. Chem. 266:16948-16953. Hepsin is thought to play a role in cell growth and is known to be produced at a particularly high level in the liver, as well as in human hepatoma cells, some other cancer cells and nerve cells. See, e.g., Torres-Rosado et al., supra. For further information regarding hepsin and its correlation to prostate cancer, see, e.g., Kurachi et al. (1994) Methods in Enzymology 254:100-115; Hooper et al. (2001) J. Biol. Chem. 276:857-860; and Welsh et al. (2001) Cancer Research 61:5974-5978.

In the present invention, the term “hepsin” is used to refer to any portion of the hepsin protease which exhibits substantially similar cleavage patterns to an intact hepsin molecule. For example, a hepsin molecule typically comprises a transmembrane domain, a pro-domain, and a catalytic domain. However, for many screening applications, a soluble form of hepsin, e.g., without the transmembrane domain, is preferred.

A “hepsin recognition site” is a peptide sequence or non-peptide moiety that is recognized and typically cleaved by hepsin. The hepsin recognition sites employed in the present invention typically comprises an amino acid sequence, e.g., about 4 to about 25 amino acids. The amino acids are typically selected to form a hepsin specific cleavage site, e.g., a sequence that is cleavable by hepsin. In addition, the sequence is preferably specific for hepsin, e.g., it is not cleaved by other proteases. The recognition site is typically a portion of a hepsin substrate, which is cleaved by hepsin upon recognition. For example, a recognition site typically comprises one or more residue to which hepsin binds prior to cleavage. Cleavage yields can range anywhere from about 0.1% to 100% cleavage of the substrate.

“Hepsin substrates” of the present invention include, but are not limited to, proteins, polypeptides, peptides, and the like. A protease, such as hepsin, catalyzes the hydrolysis of a hepsin substrate, e.g., a protein or polypeptide, producing degraded protein products. Hepsin substrates as provided herein are molecules, e.g., peptide based molecules, that are cleavable by hepsin. In the present invention, hepsin substrates also include non-peptide substrates as well as substrates comprising a peptide attached to a non-peptide moiety. For example, a coumarin-based substrate comprising an amino acid and a non-peptide coumarin moiety optionally serves as a hepsin substrate. Such novel substrates are optionally used to further explore the specificity of hepsin.

In some embodiments of the present invention, the hepsin substrates comprise Pₙ . . . P₄ P₃ P₂ P₁ P₁′ P₂′ P₃′ P₄′ . . . Pₙ′. As used herein, the nomenclature for substrates refers to prime side and non-prime side positions, wherein each Pₙ and Pₙ′ (alternatively referred to as P₋ₙ) is typically a substrate component or moiety, such as an amino acid or amino acid mimetic. Cleavage, e.g., amide bond hydrolysis, typically occurs between P₁ and P₁′ (see, e.g., Schechter and Berger (1968) Biochem. Biophys, Res. Commun. 27:157-62). For example, hepsin typically cleaves an amide bond between two substrate moieties, such as between an amino acid in a prime side peptide P₁ position and an amino acid in a non-prime side peptide P₁′ position. Optionally, “n” ranges from zero to 21 substrate moieties, thereby providing substrates ranging from 4 to 25 units (e.g., amino acids) in length.

In other embodiments, the substrates comprise Pₙ . . . P₄ P₃ P₂ P₁X, wherein X is a selected component such as a peptide, a protein, a cell modulating reagent such as a cytotoxic reagent, a label moiety, a therapeutic moiety, or the like. For example, in some embodiments, hepsin cleaves a substrate between P₁ and X, wherein P₁ is a peptide moiety (e.g. an amino acid), and X is a diagnostic moiety such as a coumarin compound which fluoresces upon release from the peptide.

A peptide or substrate of the invention is “cleavable by” hepsin if, when mixed with a hepsin molecule, the substrate or peptide is cleaved, e.g., at a cleavage site as described above, e.g., between the P₁ and P₁′ positions or between P₁ and X. The substrates of the invention typically comprises a non-prime side sequence (e.g., to the N-terminal side of the cleavage site) and an additional moiety, e.g., a prime side sequence (e.g., to the C-terminal side of the cleavage site), a therapeutic or diagnostic moiety, such as a cytotoxin or fluorophore. When a substrate molecule is cleaved by hepsin, the additional moiety is released from the peptide upon cleavage (unless the additional moiety is coupled to the substrate molecule at a second position distal from the cleavage site).

Hepsin substrates of the present invention include, but are not limited to, tetrapeptide sequences in which P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; and P₄ is arginine, lysine, proline, valine, leucine, isoleucine, methionine, norleucine, alanine, glycine, tryptophan, phenylalanine, or tyrosine. Optionally, the amino group of the N-terminal amino acid (e.g., P₄) is derivatized or blocked; preferably, the N-terminal amino acid of the tetrapeptide (or of a peptide having n amino acids) is N-acetylated. Preferably P₄ is selected from the group consisting of arginine, lysine, proline, valine, leucine, and alanine. Preferred peptides for use in the hepsin-cleavable molecules of the present invention include KRLR, KQLR, PQLR, RQLR, RRLR, PRLR, PKLK, PKLR, and PRLK.

In addition to the above described peptide sequences, the hepsin cleavable molecules of the present invention typically comprise an additional component X, wherein X comprises a cell modulating moiety, a label moiety, a polypeptide (e.g., comprising from about 1 to about 25 amino acids, such as the a prime-side coupled peptides described herein), or a non-native or non-naturally occurring peptide sequence, e.g., one not found in a naturally-occurring hepsin substrate. Other X components that are optionally included in the hepsin substrates of the invention include, but are not limited to: polyalcohols such as polyethylene glycol, biotin, various carbohydrates or carbohydrate polymers, or crosslinking agents. The X component can be coupled or attached to the hepsin cleavable molecule at either or both of the P₁ and P₄ moieties. In some embodiments, the hepsin cleavable molecules are provided in the format P₄P₃P₂P₁X and are cleavable by hepsin between the P₁ moiety of the peptide sequence and the X component.

In some embodiments, component X comprises a prime-side peptide or peptide-like sequence (the units of which are designated Pₙ′, or sometimes P₋ₙ). For example, a hepsin cleavable molecule or hepsin substrate of the invention optionally comprises a non-prime side sequence and a prime side sequence as described above (e.g. Pₙ . . . P₄P₃P₂P₁P₁′P₂′P₃′P₄′ . . . Pₙ). Preferred prime sequences are those in which P₁′ is methionine, norleucine, leucine, isoleucine, valine, alanine, tyrosine, or threonine; P₂′ is alanine, phenylalanine, tyrosine, threonine, histidine; P₃′ is arginine, lysine, histidine, glutamine, serine, threonine, tyrosine, tryptophan, glycine, leucine or methionine; and P₄′ is aspartic acid, glycine, proline, valine, or methionine.

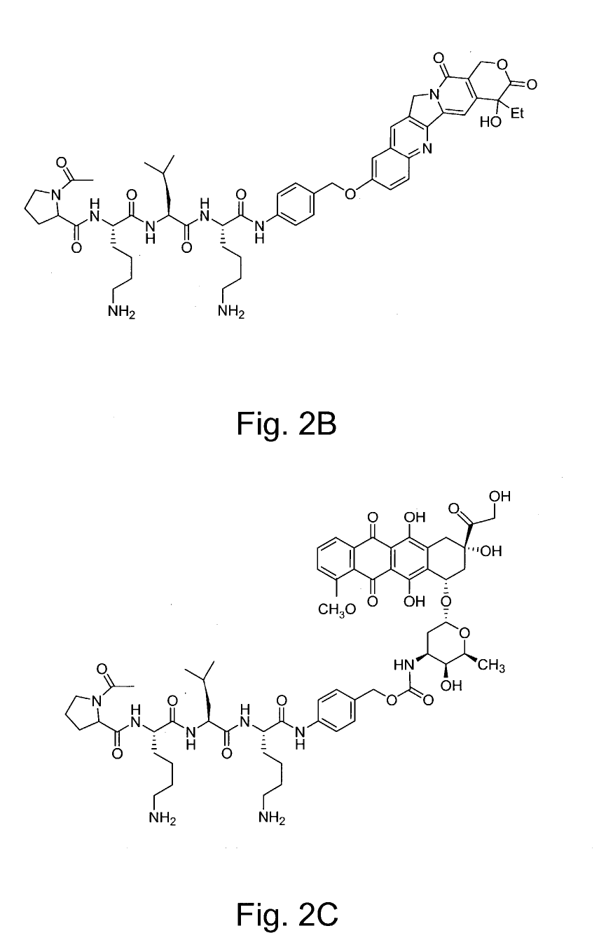

In a further embodiment, component X comprises a cell modulating factor (e.g., a compound or moiety that affects cellular function and/or activity). Exemplary cell modulating factors for use in the present invention include, but are not limited to: cytotoxic moieties, antiproliferative moieties, anti-metastatic moieties, apoptosis-inducing moieties, necrosis-inducing moieties, and the like. Cytotoxin moieties include, but are not limited to, doxorubicin, daunorubicin, epirubicin, idarubicin, anthracycline, paclitaxel, camptothecin, mitomycin C, and/or phenylenediamine mustard, bacterial toxins, and/or the like. Examples of hepsin cleavable molecules comprising a non-prime side peptide moiety and a prime-side cytotoxic moiety are provided in FIGS. 2A, 2B and 2C.

Once a hepsin substrate sequence is determined, e.g., from a positional scanning library as described below or by other methods known in the art, the substrate peptides of the present invention are typically synthesized using any recognized procedure in the art, e.g., solid phase synthesis, e.g., t-boc or fmoc protection methods, which involve stepwise synthesis in which a single amino acids is added in each step starting with the C-terminus. See, e.g., Fmoc Solid Phase Peptide Synthesis: A Practical Approach in the Practical Approach Series, by Chan and White (Eds.), 2000 Oxford University Press. The peptides are then optionally used to provide substrates, inhibitors, prodrugs, diagnostics, etc. as described below.

In forming the various prodrugs, diagnostics, inhibitors, and the like, the peptide sequences provided herein are optionally linked to non-peptide moieties, e.g., aldehydes, cytotoxic compounds, labels, or other additional components. Such non-peptide moieties are typically coupled to the peptide sequences, either directly, e.g., via a covalent bond (such as an amide bond or carbamate linkage), or indirectly via a linker molecule (such a glycol linker or Rink linkers, which are described in more detail below).

Hepsin Substrate Libraries

For many screening applications, e.g., screens for hepsin activity or hepsin substrate specificity profiles, a library of substrates or putative substrates is desired. A “library” is a collection or group of molecules, e.g., about 350-400 or more molecules, about 1000 or more molecules, about 10,000 or more, and/or about 100,000 or more molecules. As used herein, the term “about” typically refers to a variation in value of +/−20%, or preferably +/−10% or +/−5%, or in some embodiments +/−1%. Typically, each member of the library comprises a different molecule. As such, the number of members in a given library of the present invention is optionally the number of constitutive components, or substrate moiety options (e.g., 19-20 amino acid options), to the power of how many positions are being varied (e.g., 3 positions in a 1-fixed-position tetrapeptide). For example, a library of tetrapeptide substrates generated using 20 amino acids and keeping the P₁ position fixed as lysine can comprise a maximum collection of (20)³ or 8,000 different molecules e.g., different peptide sequences that are potentially cleavable by hepsin.

A library of putative hepsin substrates is a library or collection of molecules that may or may not be cleavable by hepsin, e.g., their ability to be cleaved by hepsin is yet to be determined. In the present invention, such a library is used, e.g., to probe substrate specificity. The molecules are believed to be, or are constructed to be, cleavable by hepsin, but are typically developed for testing to determine which ones are actually cleavable by hepsin.

These libraries are optionally used to provide non-prime side information regarding the enzyme active site with respect to the various member substrates of the library. For example, a non-prime substrate sequence, e.g., the first four amino acids on the non-prime side (e.g., N-terminal side) of the cleavage site are identified as optimal, e.g., for hepsin. This information is optionally used to design more selective and/or potent substrates. For example, different fluorogenic compounds are optionally employed to increase the sensitivity (e.g., detection sensitivity) of these substrates. The substrates identified also can provide valuable diagnostics for the identification of protease activity in complex biological samples, and are valuable in screening efforts to identify protease inhibitors. For example, the optimal non-prime information is optionally used to design more selective and/or potent inhibitors (e.g., inhibitors that serve as therapeutic agents or biological tools), to bias the generation of libraries aimed at identifying prime side specificity determinants, and/or to provide panning information that allows for the generation of specific substrates and inhibitors in the context of an entire set of proteases. This provides a genomic approach rather than a target-based approach.

The libraries are typically created using peptide synthesis techniques well known to those of skill in the art, or the techniques described in international patent application PCT/US02/27357, filed Aug. 27, 2002, entitled “Combinatorial Protease Substrate Libraries,” by Backes et al. For the varied positions, a mixture of amino acids is added to the coupling reaction, e.g., to couple a random substrate moiety or amino acid to a support-bound coumarin molecule. The mixture of amino acids can be a combination of all 20 amino acids; alternatively, the mixture can be a subset of amino acids, include derivatized or blocked amino acids, and the like. Furthermore, the amino acids can be provided in equimolar ratios, or in varied amounts as desired. In addition, the libraries are optionally created using non-peptide molecules in the P₁, P₂, P₃, and/or P₄ positions.

The term “substrate moiety” refers to a component of the substrate molecule, and as such includes any amino acid or amino acid mimetic, as well as the labels, cell modulating factors, cytotoxic compounds and inhibitors described herein, and other components of interest. The substrates and/or putative substrates of the present invention typically comprise from about 1 to about 15 substrate moieties, or from about 4 to about 25 substrate moieties. In addition, selected components are optionally coupled to or linked to the substrates. Such selected components include, but are not limited to: peptides, proteins, non-peptide moieties, sugars, polysaccharides, polyethylene glycol, small molecules, organic molecules, inorganic moieties, label moieties, therapeutic moieties, and/or the like. For example, a fluorogenic compound, such as a coumarin, is optionally coupled to a peptide to form a hepsin substrate. Alternatively, the selected component coupled to a hepsin substrate of the invention is a quantum dot, a cytotoxic moiety, a detectable label, a prodrug moiety, or the like.

Typically, the substrate moieties and selected components, when used in a substrate or putative substrate, form a hepsin cleavage site or a potential hepsin cleavage side, e.g., hepsin cleaves between two of the substrate moieties, such as between two amino acids or between an amino acid and a coumarin moiety. In some embodiments, the substrate moieties comprise amino acids which provide prime side and/or non-prime side specificity to a hepsin cleavage site. In other embodiments, labels that allow for detection of a cleavage event are incorporated into the substrates of the invention.

In some embodiments of the present invention, the hepsin substrate libraries or putative substrate libraries of the invention comprise a plurality of peptides, wherein one or more positions in the peptide sequence is held constant and the others are varied. These libraries, also known as positional scanning libraries, are described in more detail below, along with their use in determining substrate specificity, e.g., prime side and non-prime side specificity.

A positional scanning library, e.g., for protease substrates, is optionally created to probe the prime and/or non-prime specificity of hepsin. Such libraries are another aspect of the present invention. As one example, four 20-well sub-libraries are optionally created, wherein each of the four sub-libraries has a different fixed amino acid position, e.g., P₁, P₂, P₃, or P₄. For example, in a first sub-library, each of the twenty wells contains a library of substrates wherein P₁ is fixed at one of twenty different amino acids, while the other positions, P₂, P₃, and P₄, are varied. In some of the embodiments of the present invention, the libraries contain about 6859 different substrates per well (i.e., one fixed position and three variable positions per substrate, and using 19 different amino acids during generation of the library, cysteine having been excluded from the synthesis mixture).

Additional sub-libraries are also optionally created, e.g., with two fixed positions, e.g., P₁/P₂, P₁/P₃, P₁/P₄, P₂/P₃, P₂/P₄, or P₃/P₄. This produces six sub-libraries of 400 wells each (representing each possible combination of the two fixed elements, and the 20 possible elements in each of the fixed positions), wherein each well contains about 361 different substrate sequences (e.g., using the 19 amino acids in the two variable positions). Therefore, the libraries of the invention typically involve about 2400 wells total and the libraries contain well over 100,000 different substrates, e.g., coumarin based substrates. The preferred amino acid for each position, e.g., in a hepsin substrate, is optionally determined using these positional scanning libraries. See, e.g., Harris et al. (2000) Proc. Natl. Acad. Sci USA 97:7754-7759 for a general description of how such libraries are used to determine optimal substrate sequences.

A non-prime side positional scanning library is typically constructed using a detectable moiety, e.g., a moiety that is not detectable until after it has been cleaved from the substrate (e.g., the peptide). For example, the substrate of a non-prime side scanning library optionally comprise the following:

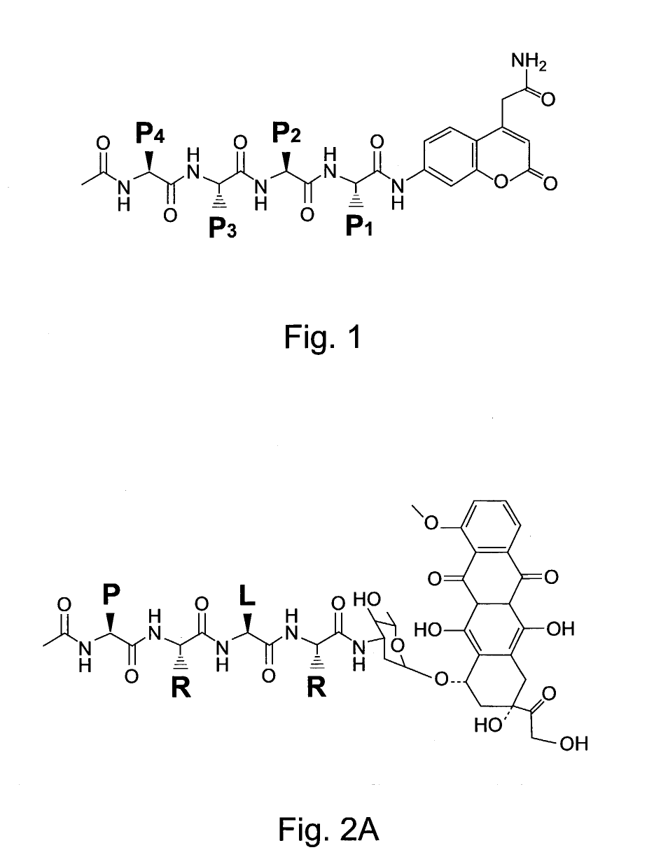

P₄P₃P₂P₁X

wherein P₄-P₁ comprise amino acids or amino acid mimetics randomized as described above and X comprises a detectable moiety, such as coumarin. An example library member structure for use in a positional scan library for analysis of non-prime specificity is provided in FIG. 1.

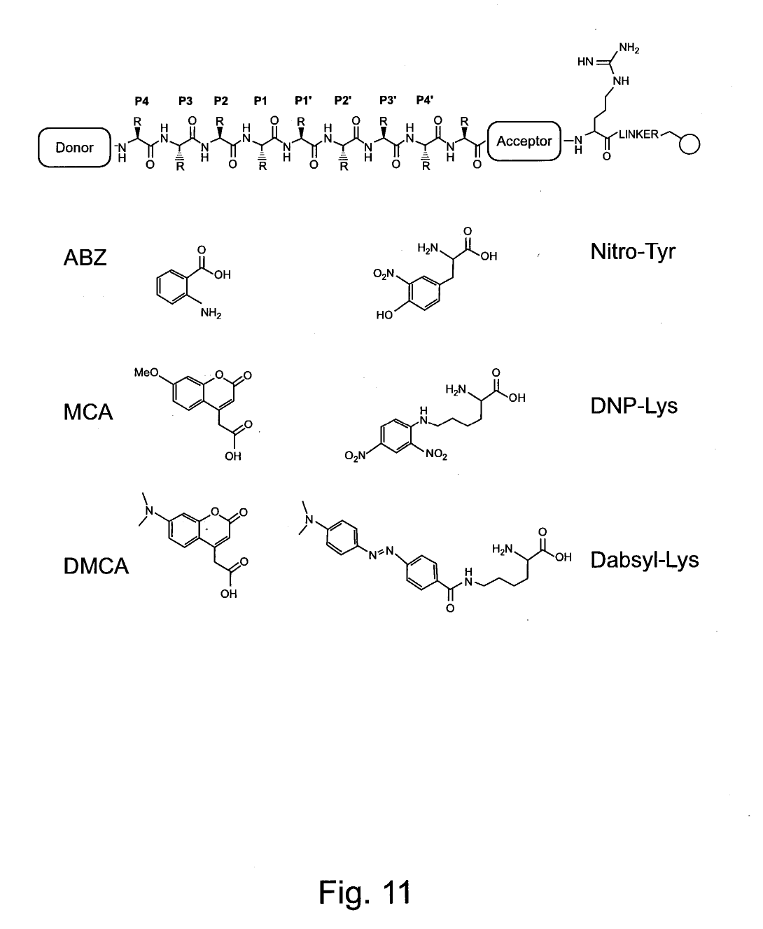

Optionally, prime side specificity can also be analyzed or probed using putative substrate libraries of the present invention. In a preferred embodiment, a prime side position library, e.g., for determining prime side substrate specificity, is constructed using a donor moiety, an acceptor moiety, and a preselected non-prime substrate sequence. Donor moieties and acceptor moieties in the present invention typically comprise fluorescence resonance energy transfer pairs, such as those depicted in FIG. 11. A typical donor moiety for use in the present invention absorbs light at one wavelength and emits at another wavelength, typically a higher wavelength. The acceptor moiety of the invention typically absorbs at the wavelength of either the absorption or emission wavelength of the donor moiety. For example, the acceptor is used as a quencher for the donor moiety. However, the acceptor typically only quenches the absorption or emission of the donor when the two are in proximity, either in high concentrations or when tethered to each other, e.g., chemically bonded. The donor-acceptor pairs are then used to detect protease cleavage, e.g., hepsin cleavage, of the substrates of the libraries in the present invention. For example, when cleavage occurs, the acceptor no longer quenches the signal of the donor.

One or more prime position substrate moiety is typically coupled to an acceptor moiety. The prime substrate moieties typically comprise amino acids or amino acid mimetics which are used to form a hepsin cleavable molecule. In a typical library, about four substrate moieties are coupled to the acceptor, e.g., P₁′, P₂′, P₃′, and P₄′. However, the number of substrate moieties coupled to the acceptor is optionally varied, e.g., from about 1 to about 15, but is more typically, about 2 to about 6, and most typically four. Typically, the substrate moieties are coupled to an acceptor using standard peptide synthesis techniques, e.g., Fmoc synthesis.

After the prime side positional substrate is coupled to the acceptor, a preselected non-prime substrate, e.g., an optimal or preferred non-prime sequence that has been identified as described above, is coupled to the prime position substrate. “Preselected substrate moieties” are determined as described above, and in PCT/US02/27357 by Backes et al, supra, using, for example, a positional scanning library. The preselected sequences are typically about 2 to about 20 substrate moieties, e.g., amino acids, in length, more typically about 2 to about 6, and most typically about 4 amino acids or substrate moieties in length. In the present invention, preselected non-prime side substrate sequences include, but are not limited to, the tetrapeptides KRLR, KQLR, PQLR, RQLR, RRLR, PRLR, PKLK, PKLR and PRLK, although other peptide based hepsin substrates as described herein are also considered.

Typically, a non-prime optimal or preselected sequence is identified by methods well known to those of skill in the art. The non-prime sequence information is then used to bias the composition of a donor-quencher construct in a positional scanning format to obtain prime-side substrate specificity information. In essence, the non-prime information gathered in a first profiling experiment is used to fix the catalytic register of a second library, e.g., a donor-quencher library, thus reducing the total number of variable library positions. As a consequence, the complexity of the donor-quencher library is vastly reduced allowing for straightforward interpretation of prime side profiling results. In this manner, a complete substrate profile is obtained. The complete substrate profile conveniently provides optimal substrate compositions, e.g., amino acid or non-peptide sequences, for both sides of an enzyme cleavage site, as well as kinetic data. However, positional scanning of the prime-side without bias in the non-prime side sequence is also contemplated in the present invention.

Once one or more non-prime sequences, e.g., optimal or preferred sequences, are selected or identified (e.g., by using standard native sequences or performing a positional non-prime scan) a library of substrates is constructed. Libraries are optionally constructed using 1, 2, 3, or more fixed positions. For example, substrates are optionally created in which more than four positions are provided and profiled on each side of the cleavage site. More than one preselected non-prime sequence is optionally used to create multiple libraries to scan the prime side of the cleavage site, e.g., to obtain more complete profiling results. Once the libraries are created, they are analyzed as described below to determine optimal prime side substrate moieties or amino acids.

After a preselected non-prime positional substrate sequence has been added to the prime position substrate/acceptor moiety, a donor is coupled to the preselected non-prime substrate. The donor typically comprises one member of a FRET pair as described above, e.g., aminobenzoic acid, 7-methoxy-4-carbamoylmethyl coumarin, 7-dimethylamino-4-carbamoylmethyl coumarin, or the like. In alternate embodiments, the donor moiety is coupled to the prime side substrate and the acceptor moiety is coupled to the preselected non-prime substrate.

For example, a substrate for use in a prime position library is typically made by coupling an acceptor moiety, e.g., a FRET acceptor, to a solid support, e.g., a polystyrene or polypropylene resin. Acceptors of the invention include, but are not limited to, nitro-tyrosine, dinitrophenol-lysine, dabsyl-lysine, and the like. Other solid supports available include, but are not limited to, polyacrylamide, polyethylene glycol, and the like. In some embodiments, the acceptor is coupled to the solid support via a linker, e.g., an arginine linker. Rink linkers, glycol linkers, or any other linker moiety typically used in peptide synthesis protocols are also optionally used. A donor is then coupled to the preselected non-prime substrate. Exemplary donor/acceptor pairs include, but are not limited to, aminobenzoic acid and nitro-tyrosine, and others that are well known to those of skill in the art. FIG. 11 illustrates exemplary donor/acceptor pairs for use in the present invention, as well as an example of a prime side scan library member. A plurality of substrates prepared in this manner provides a library tailored to a specific protease, e.g., hepsin. By coupling the preselected non-prime substrate directly to the prime side substrate, the cleavage site is set.

Library Screening and Substrate Specificity Profiling Methods

In brief, the methods typically comprise profiling a substrate library, e.g., a coumarin-based substrate library. Techniques known in the art are then used to reveal an optimal substrate sequence for the non-prime positions of a substrate of interest or a first library of substrates. Next, a second library is prepared, e.g., a prime side scan library. Typically, a library for a prime scan (e.g., a library for probing prime side substrate sequence specificity) is prepared using a donor-acceptor pair and the optimal non-prime sequences obtained, e.g., as described above. The prime side scan library is then incubated with the enzyme of interest and monitored to determine one or more optimal prime substrate sequence.

For example, a typical method comprises providing a library of putative hepsin substrates, each of which comprises a putative hepsin recognition site and incubating the library with hepsin. The substrate profile is obtained by monitoring cleavage of the putative hepsin substrates by the hepsin, thereby providing a substrate profile hepsin.

A library of substrates, e.g., as described above, is typically incubated with an enzyme of interest, to determine substrate specificity. For example, a library created with one or more non-prime substrate moiety tailored to hepsin substrates is used to create a library to identify prime side hepsin substrate sequences. Therefore, such a library would be incubated with hepsin. The enzyme is added to the library, which has typically been released from the solid support. For example, for a library comprising 600 microwells with multiple sequences in each, enzyme is added to each of the wells.

Fluorescence is typically detected at multiple time points in the course of the enzymatic reaction, e.g., continuously, or at a single time point at or near the end of the reaction. By continually monitoring the fluorescence in each well of the library, kinetic data is also optionally obtained. The detection is used to monitor which wells, e.g., which substrates are cleaved by the enzyme.

The present invention provides methods of screening a library of compounds for a modulator of hepsin activity. The screening methods include the steps of (a) providing a first library comprising a plurality of putative hepsin substrates having a structure P₄P₃P₂P₁X, wherein P₁, P₂, P₃ and P₄ comprise substrate moieties at non-prime positions and X comprises a label moiety; (b) analyzing the first library to identify substrate moieties at one or more non-prime positions that result in cleavage of the putative hepsin substrate between P₁ and X by a hepsin protease; (c) constructing a second library comprising the identified substrate moieties; (d) incubating the second library with the hepsin protease; and (e) monitoring fluorescence resonance energy transfer between the members of the FRET pair, to identify one or more optimal prime substrate moieties, thereby providing the substrate profile for the enzyme.

As described herein, the hepsin-cleavable substrates such as those employed in the second library can be labeled with a variety of label moieties. In one embodiment, constructing the second library typically involves (i) coupling a first member of a fluorescence resonance energy transfer (FRET) pair to a substrate moiety on an N-terminal side of a putative hepsin cleavage site, wherein the substrate moiety comprises an identified substrate moiety from the first library; (ii) coupling a second member of the FRET pair to a substrate moiety on a C-terminal side of the putative hepsin cleavage site; and (iii) linking the compounds of (i) and (ii) together to form members of the second library.

Optional FRET pairs for use in the screening methods include, but are not limited to, amino benzoic acid and nitro-tyrosine; 7-methoxy-4-carbomoylmethylcoumarin and dinitrophenol-lysine, or 7-dimethylamino-4-carbomoylmethylcoumarin and Dabsyl-Lysine. Furthermore, the substrate moiety X on the C-terminal side of the hepsin cleavage site (i.e., the prime side substrate moieties) optionally comprises a tetrapeptide, such as P₁′P₂′P₃′P₄′, wherein P₁′ is attached to P₁ and is methionine, norleucine, leucine, isoleucine, valine, alanine, tyrosine, or threonine; P₂′ is alanine, phenylalanine, tyrosine, threonine, or histidine; P₃′ is arginine, lysine, histidine, glutamine, serine, threonine, tyrosine, tryptophan, glycine, leucine or methionine; and P₄′ is attached to the label moiety and is aspartic acid, glycine, proline, valine, or methionine.

Furthermore, the present invention provides methods of obtaining a substrate profile for a hepsin activity. The method includes the steps of (a) providing a library of putative hepsin substrates, each of which comprises a putative hepsin recognition site, (b) incubating the library in the presence of the hepsin; and (c) monitoring cleavage of the putative hepsin substrates by the hepsin, thereby providing the substrate profile for the hepsin. Preferably, the putative hepsin recognition sites of the member putative hepsin substrates comprises one or more non-prime positions and one or more prime positions, each of which positions is occupied by a substrate moiety. As described herein, the prime and non-prime positions flank the putative hepsin cleavage site.

In some embodiments of the methods, the substrate moieties that occupy one or more of the non-prime positions are preselected to allow cleavage of the substrate at the putative hepsin cleavage site by the hepsin, while allowing the moieties on the prime-side of the cleavage site to vary. Alternatively, both the substrate moieties that occupy the non-prime and the prime positions vary among different members of the library of hepsin substrates (e.g., no pre-selection of library members).

In a preferred embodiment, the putative hepsin substrates further comprise a label moiety. Optionally, the label moiety is a molecule with fluorescent properties which alter upon cleavage from the substrate, or a matched donor: acceptor pair of fluorescence resonance energy transfer (FRET) compounds. In one embodiment, a fluorescence donor moiety and a fluorescence acceptor moiety are attached to the putative hepsin substrate library members on opposite sides of the putative hepsin cleavage site, such that monitoring the cleavage of the putative hepsin substrates is performed by detecting a fluorescence resonance energy transfer. Monitoring can include detecting a shift in the excitation and/or emission maxima of the fluorescence acceptor moiety, which shift results from release of the fluorescence acceptor moiety from the putative hepsin substrate by the hepsin activity.

Optionally, the one or more non-prime positions of the putative hepsin substrates employed in the methods include a tetrapeptide sequence. Exemplary tetrapeptide sequences include, but are not limited to, KRLR, KQLR, PQLR, RQLR, RRLR, PRLR, PKLK, PKLR and PRLK.

Non-Prime Side Positional Scan for Hepsin Substrate Specificity

Typically, to obtain a complete substrate profile for an enzyme, e.g., a protease, a non-prime scan and a prime scan are performed. A “non-prime scan” refers to the scanning library used to determine an optimal substrate sequence for the non-prime side of the cleavage site and/or the results of an analysis of that library. A “prime side scan” refers to the opposite side of the cleavage site, either the library used to probe those positions or the results of such a probe.

Non-prime scanning libraries are known to those of skill in the art (see, e.g., Harris et al., supra). For example a coumarin-based library is used to determine an optimal amino acid sequence for the non-prime sequence for thrombin substrates. See, e.g., FIG. 1, illustrating an example substrate for a non-prime scan library. The substrate shown comprises a coumarin compound and four substrate moieties or residues, e.g., P₁, P₂, P₃, and P₄.

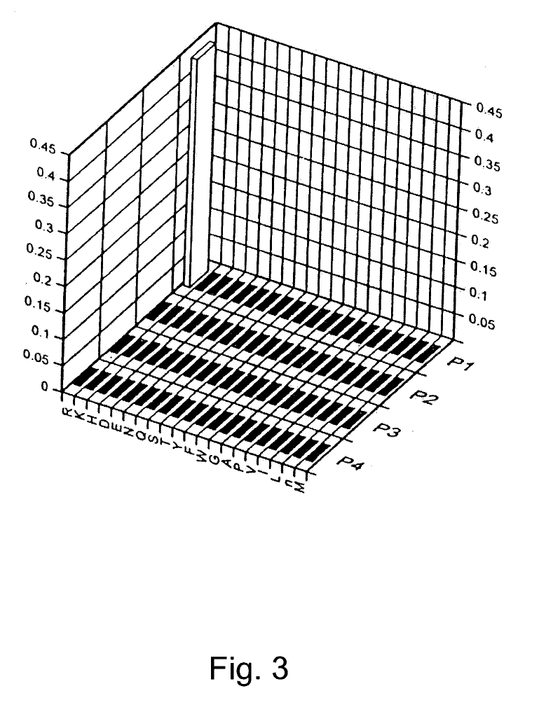

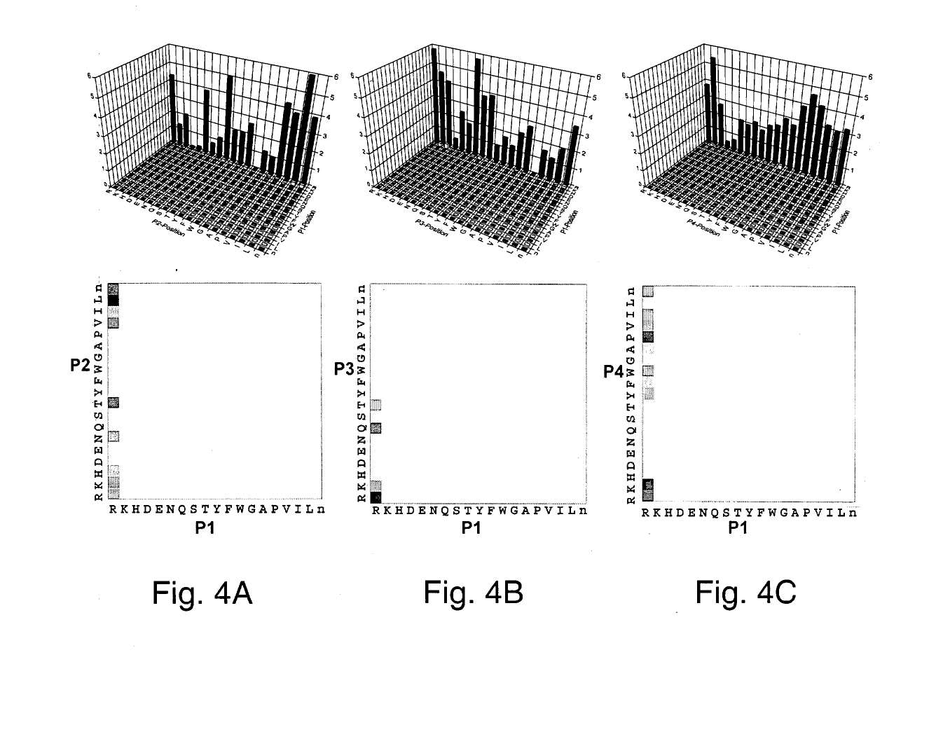

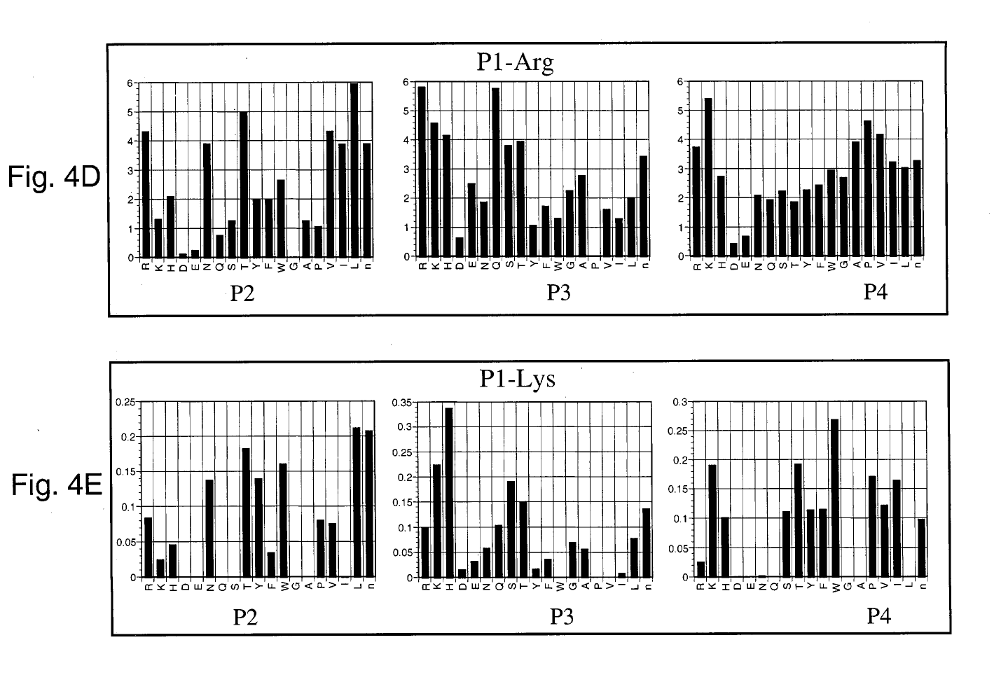

FIGS. 3 and 4 provide data obtained from incubating a non-prime scan library of coumarin-based substrates with hepsin. The 3-dimensional histograms depict the enzyme activity (as indicated on the z axis) for pools of library members having, in FIG. 3, a single “fixed” position, or in FIG. 4, two “fixed” positions (depicted by the x/y axes) in the tetrapeptide-coumarin substrate. When hepsin acts on a substrate, the substrate is cleaved between P₁ and the coumarin moiety, thereby releasing a fluorogenic coumarin moiety, which is detected. As shown in FIG. 3, arginine is an optimal P₁ residue. FIG. 4, Panels A-C provides 3D histograms and corresponding 2-dimensional “signal intensity” plots illustrating preferred residues for positions P₂-P₄, based on having a fixed P₁ substituent. The data for the rows in which P₁ is set to arginine or lysine are expanded in Panels D and E, respectively (note the difference in scale). For example, the preference in the P₂ position is for large aliphatic amino acids, valine, leucine, isoleucine and methionine as well as basic amino acids, arginine, lysine and histidine and polar amino acids, asparagines and threonine. The P₃ position prefers basic amino acids as well as the polar amino acids, glutamine, serine and threonine. The P₄ position also prefers basic amino acids, but can also accommodate the majority of hydrophobic and aliphatic amino acids.

To provide a complete substrate profile of an enzyme, a non-prime side scan is typically performed to obtain one or more preferred and/or optimal non-prime substrate sequence. Such an analysis is referred to herein as “positional scanning.” See also, Rano et al. (1997) Chem. Biol. 4:149-155. In the present invention, the optimal non-prime substrate moieties identified are typically used to create, e.g., a second library, which is used to probe the prime side substrate specificity. In this way, complete profiles of substrate specificity are determined.

Prime Side Positional Scan for Hepsin Substrate Specificity

To further probe substrate specificity of an enzyme by providing prime as well as non-prime specificity information, a second library is optionally created, e.g., in addition to the non-prime side substrate library described above, that is used to probe non-prime substrate specificity, and from which a non-prime sequence is preselected. The prime position substrates and libraries provided herein take advantage of information obtained from a non-prime scan, e.g., to provide preselected non-prime substrate sequences. For some enzyme system, this analysis can preferably be performed in reverse order, by generating the prime-side data profile and then determining the non-prime side specificities, or by not taking advantage of the first scan in generating the compounds for inclusion in the second library.

To determine a prime substrate specificity profile for hepsin, a donor-acceptor library is typically used. For example, a methoxy coumarin positioned at the C-terminus of the peptide is optionally used as a donor, with a dinitrotyrosine at the N-terminus of the substrate as the acceptor. Optionally, the donor and acceptor moieties need not be positioned at the termini of the substrate molecule, as long as the signal generated by the pair is altered upon cleavage of the substrate by the protease (e.g., one is released from the molecule). A preselected sequence is used for the non-prime side of the substrate while the prime side sequence is varied. For example, the non-prime side of the substrate in a substrate library of the invention is optionally kept constant as the sequence determined from a coumarin library, P₄-Arg, P₃-Lys, P₂-Leu, P₁-Arg, or any other sequence as provided above. The prime-side four amino acid positions are typically randomized as all 20 natural amino acids. However, in some embodiments, norleucine is optionally used to replace methionine and/or cysteine is optionally excluded.

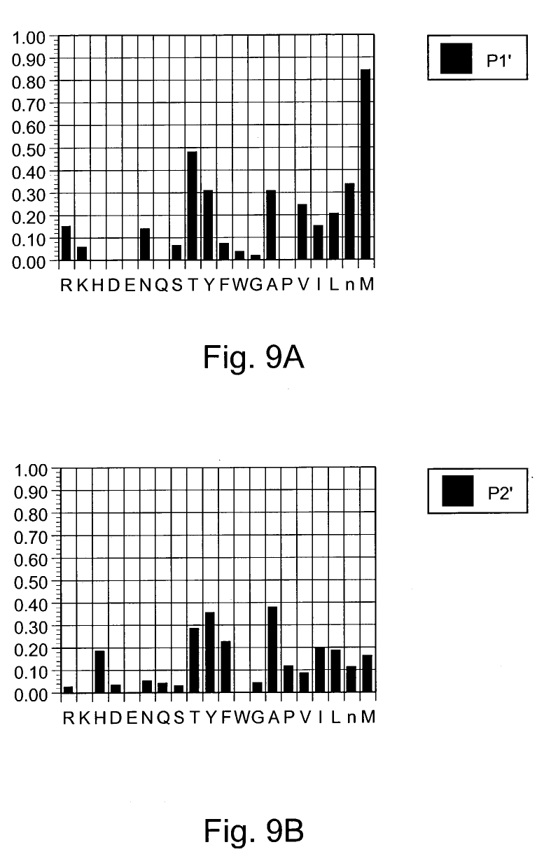

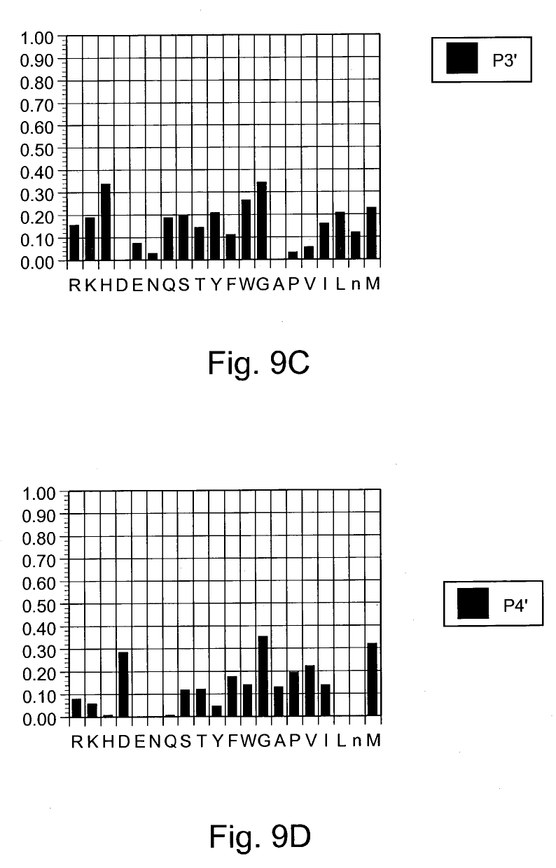

An exemplary specificity profile, with the preselected sequence being P₄-Arg, P₃-Lys, P₂-Leu, P₁-Arg, is provided in FIG. 9. Panels A-D provide prime side substrate specificity for P₁′, P₂′, P₃′ and P₄′ with the y-axis representing relative fluorescence units per second and the x-axis representing the amino acid held constant in the substrate.

Prime and non-prime information, e.g., determined as described above, is optionally used to search genomic databases, e.g., for similar cleavage sites in proteins and provide possible macromolecular substrates that are key to the biological function of hepsin. In addition, the information is used to design peptide based inhibitors of hepsin and prodrugs and diagnostic reagents based on hepsin specificity.

In another embodiment, the present invention provides databases constructed using the above substrate profile information. These data bases are optionally used in the applications described above, e.g., to design improved hepsin substrates, for use in identifying hepsin inhibitors, and/or for use in characterizing hepsin, an enzyme for which substrates were previously unknown or incompletely characterized.

A database of the invention typically comprises records for members, e.g., each member of a library of putative hepsin substrates, e.g., the libraries described herein. Each record typically comprises information regarding the identity of a substrate moiety or group of substrate moieties, e.g., amino acids, peptides, or non-peptides, that occupy each of one or more prime and non-prime positions of a particular putative hepsin substrate. Data from assays used to determine the ability of hepsin to cleave the putative hepsin substrate is also included in the database, as well as kinetic data obtained from the assay, e.g., by detecting at multiple time points in the course of a reaction.

The prime and non-prime information is also optionally used to design more selective and potent substrates, e.g., for use as therapeutic agents or biological tools. Multiple fluorogenic compounds can be employed with the determined amino acid specificity sequence to increase the sensitivity and efficacy of these substrates for a particular system.

Furthermore, substrates of the present invention are valuable as diagnostics for the identification of protease activity in complex biological samples and for screening efforts to identify protease inhibitors. The overall strategy when applied, e.g., to an entire class of proteases, provides panning information that allows for the generation of specific substrates and inhibitors in the context of an entire protease class. The non-prime and prime specificity information can be employed to bias bead-based and phage display methods, to design cleavage sites in fusion proteins or other protein constructs, and to design prodrugs in which the protease target releases an active drug. These are described in more detail below.

Hepsin Expression Systems

To screen a large collection of compounds, e.g., libraries of putative hepsin substrates as described herein, significant amounts of active enzyme are typically used. For example, the libraries described above are optionally screened, e.g., in a high throughput system, to determine optimal hepsin substrate sequences or in search of activators or inhibitors of hepsin.

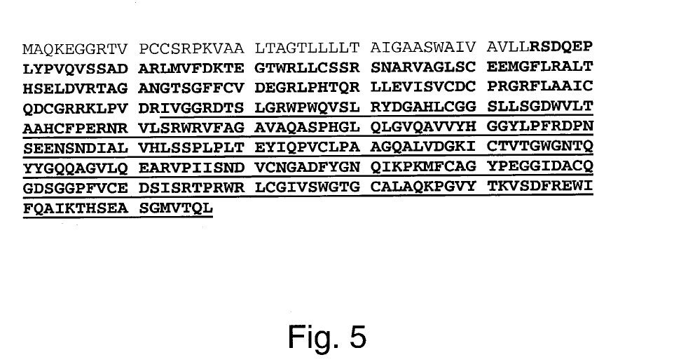

Hepsin belongs to the family of Type II transmembrane serine proteases. The protein typically comprises a secretion signal, a single transmembrane domain, a pro-domain, and a catalytic domain as shown in FIG. 5. For high-throughput screening of compound collections and/or crystallization, a soluble form of hepsin, e.g., without the transmembrane domain, is preferred. Therefore, the present system typically provides vectors for expressing a hepsin sequence without the transmembrane region.

The expression vectors of the invention typically comprise the following operably linked components: a promoter active in a selected cell type, such as insect cells; a polynucleotide encoding a secretion signal polypeptide appropriate for the selected cell type; and a polynucleotide encoding the hepsin polypeptide of interest. The hepsin polypeptides typically comprises a hepsin catalytic domain and prodomain, and optionally lack a transmembrane domain. A typical secretion signal polypeptide for use in the expression vectors of the present invention is a non-hepsin secretion signal polypeptide, such as a honeybee melittin secretion signal polypeptide. In addition, the expression vectors optionally further comprise a polynucleotide that encodes a tag, such as a polyhistidine tag, to facilitate purification of the hepsin polypeptide.

General Expression Systems

The vectors of the invention include, but are not limited to, expression vectors, plasmids, viruses, cosmids, phage, viral fragments, and the like, e.g., into which a recombinant nucleic acid has been added. The nucleic acid typically encodes the sequence for one or more of the hepsin domains described above, and one or more regulatory sequences, including, for example, a promoter, operably linked to the sequence. Large numbers of suitable vectors and promoters are known to those of skill in the art, and are commercially available.

The hepsin activity-encoding nucleic acid sequence in the expression vector is optionally linked to one or more appropriate promoter control sequence, e.g., to direct mRNA synthesis and/or protein expression. Examples of such promoters include, but are not limited to, LTR or SV40 promoter, E. coli lac or trp promoter, and phage lambda PL promoter; however, other promoters known to those of skill in the art are also contemplated. The vectors of the invention also optionally include appropriate sequences for amplifying expression and increasing secretion of an expressed protein. In addition, the expression vectors optionally comprise one or more marker genes to provide a phenotypic trait for selection of transformed host cells, such as tetracycline or ampicillin resistance in E. coli.

General texts which describe molecular biological techniques useful herein, including the generation and use of vectors, promoters, cloning, expression, and many other relevant topics, include Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2001 and Current Protocols in Molecular Biology, F. M. Ausubel et al., eds., and Current Protocols, a joint venture between Greene Publishing Associates, Inc. and John Wiley & Sons, Inc.).

Host cells, e.g., E. coli or baculovirus, are transduced, transformed, or transfected with the expression vectors of this invention by one or more mechanisms known in the art. The host cells are typically cultured in conventional nutrient media modified as appropriate for activating promoters, or amplifying the hepsin-encoding sequence. The culture conditions, such as temperature, pH and the like, are apparent to those skilled in the art and in the references cited above. Additional useful references for cloning and culture of cells include, including, e.g., Freshney (1994) Culture of Animal Cells, a Manual of Basic Technique, third edition, (Wiley-Liss, New York) and the references cited therein, Payne et al. (1992) Plant Cell and Tissue Culture in Liquid Systems (John Wiley & Sons, Inc. New York, N.Y.), and Atlas and Parks (eds.) (1993) The Handbook of Microbiological Media (CRC Press, Boca Raton, Fla.)

Vectors containing an appropriate hepsin sequence, are used to transform an appropriate host, which host is used to express the protein, e.g. hepsin. Examples of appropriate expression hosts especially include bacterial cells, such as E. coli, Streptomyces, insect cells such as Drosophila and Spodoptera frugiperda, etc.

Introduction of the vector into the host cell is optionally achieved by calcium phosphate transfection, DEAE-Dextran mediated transfection, electroporation, or other techniques known to those of skill in the art. See, e.g., Sambrook, Ausubel, Berger, as well as Davis et al. (1986) Basic Methods in Molecular Biology (Prentice-Hall Inc., New Jersey).

A host cell strain is optionally chosen for its ability to alter or enhance the expression of the inserted sequences or to process the expressed protein in a desired fashion. Such modifications of the protein include, but are not limited to, acetylation, carboxylation, glycosylation, phosphorylation, lipidation and acylation. Post-translational processing is also sometimes important, e.g., for correct, folding and/or function of the protein of interest, e.g., hepsin.

Host cells transformed with the vectors of the invention are optionally cultured under conditions suitable for the expression and recovery of the encoded protein, e.g., hepsin, from the cell culture. The hepsin protein or fragment thereof produced by a recombinant cell can be a secreted protein, a membrane-bound protein, or an intracellular protein, depending on the sequence and/or the vector used. As will be understood by those of skill in the art, expression vectors containing nucleic acids encoding hepsin are typically designed with signal sequences which direct secretion of hepsin, e.g., for use in high throughput screening. In the present invention, the hepsin transmembrane domain is optionally omitted from the vector to allow the protein to be secreted.

Following transformation of the host cells with the vectors of the invention, the cells are grown to an appropriate cell density, the selected promoter is induced, e.g., by temperature shift or chemical induction, and cells are cultured for an additional period. Cells are optionally harvested by centrifugation, disrupted by physical or chemical means. The resulting cell extract is retained, e.g., for collection and purification of the expressed protein of interest. The proteins are recovered and purified from the cell cultures by any of a number of methods well known in the art, including ammonium sulfate or ethanol precipitation, acid extraction, affinity chromatography, e.g., using a histidine tag, and the like.

Novel Hepsin Expression Systems

In one aspect, the present invention provides baculovirus expression systems in insect cells, which expression systems provides more efficient expression of active hepsin, e.g., as compared to E. coli expression systems. Expression in insect cells, as described below, typically results in reagent amounts (e.g., gram quantities) of soluble and active protease, e.g., greater than about 1 mg/L. Exemplary vector constructs and methods are also described below.

To provide soluble hepsin, the present invention provides hepsin expression vectors lacking the coding sequences for the transmembrane sequence. In addition, a coding sequence for a honeybee melittin secretion signal (Mel) is optionally appended to the codon for the N-terminus of a hepsin sequence, e.g., using PCR extension. Incorporation of this signal peptide into the polypeptide increases the secretion of hepsin from the cells and allows greater yields of active hepsin to be obtained.

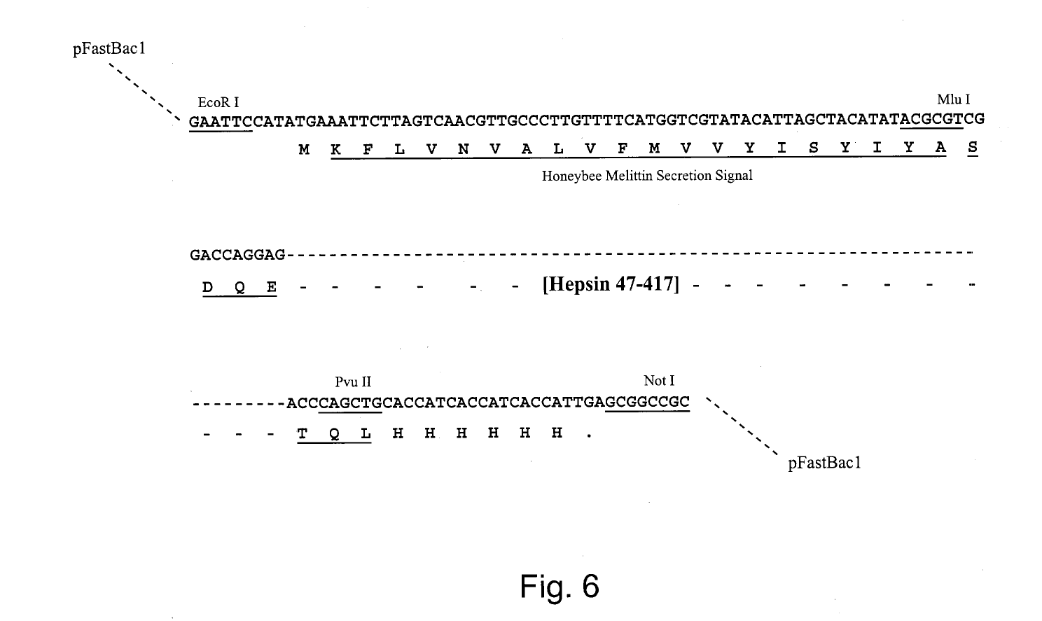

To facilitate the downstream purification, a histidine tag, e.g., a 6His tag, is optionally appended to the C-terminus of the hepsin fragments that are expressed. The hepsin-encoding constructs are typically inserted into a commercial vector such as a pFastBac1 (Invitrogen, Carlsbad, Calif.) vector, e.g., using EcoR I and Not I sites. A modified pFastBac1 vector of the invention is illustrated in FIG. 6.

Unique restriction sites, such as Mlu I and Pvu II, are also optionally added, e.g., to provide a vector that serves as a general tool for baculoviral expression of secreted proteins. Manufacturer protocols are typically followed for producing plasmids, such as those modified as described above. For example, a vector is optionally made that expresses hepsin fragments as described above, with the melittin secretion signal and with (or without) a histidine tag. Recombinant viruses carrying the vector as described are produced and then typically amplified, e.g., using techniques suggested by the manufacturer or well known to those of skill in the art.

Cells, e.g., SP9 cells, are optionally infected with recombinant virus comprising a hepsin vector as described above. The activity of hepsin in the supernatant is typically monitored, e.g., using hydrolysis of a hepsin-cleavable molecule of the present invention, such as a fluorogenic peptide, e.g., KRLR-ACC (a 7-amino-4-carbomoylcoumarin (“acc”) labeled tetrapeptide). To determine an optimal expression time, activity is optionally monitored for a number of hours after infection, e.g., about 24 to about 72 hours. The supernatant is typically collected and cleared by centrifugation. In addition, the collected hepsin is optionally purified, e.g., using a histidine tag if included, or by precipitation. Exemplary purification methods are described below. Other methods of expression and purification known to those of skill in the art are also optionally used.

Substrate Specificity Profiles

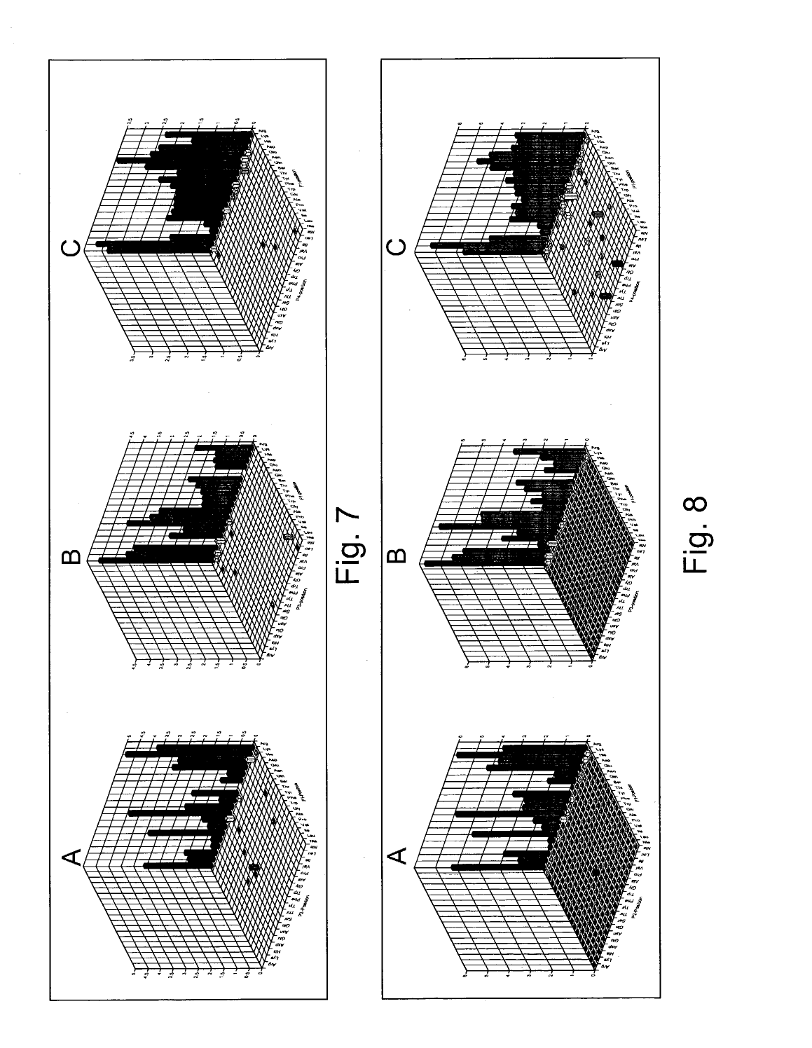

FIGS. 7 and 8 provide substrate specificity profiles obtained using the above vectors, e.g., vectors encoding hepsin fragments. FIG. 7 illustrates the results for a hepsin fragment without a histidine tag, while FIG. 8 depicts results for the same hepsin fragment with the 6-histidine tag. FIGS. 7 and 8 illustrate profile information for two position fixed substrate libraries, the two positions indicated on the x and y axes. The figures illustrate that the histidine tag does not significantly change the hepsin substrate specificity. Therefore, histidine tagged hepsin, which is more easily purified, is optionally used for screening applications, e.g., to screen for hepsin inhibitors. In addition, the profiles are consistent with a profile for hepsin produced from hepsin expressed in an E. coli expression system and refolded. Data from the refolded hepsin is shown in FIGS. 3 and 4. In all instances, the major activity is observed for hepsin substrates having either P₁-Arg or P₁-Lys, as provided above.

Hepsin Substrate Based Prodrugs

A “prodrug” is a composition that is modified to become active, often in vivo. Such compositions typically comprise a therapeutic moiety or cell modulating moiety that is cleaved from the remainder of the composition, preferably at a target site. The therapeutic or cell-modulating moiety is typically activated only after cleavage from the remainder of the composition. For example, an “anti-cancer” prodrug is one that is used in the treatment of cancer, e.g., to destroy cancer cells or tumors and/or prevent their spread into other parts of the body. The prodrugs of the invention are typically peptides linked to therapeutic moieties. The peptides are cleavable by hepsin, e.g., at cancer sites that have high concentrations of hepsin, such as those in prostate cancer. Optionally, the therapeutic moiety is a cytotoxic moiety that exhibits non-specific toxicity when released from the peptide by cleavage, e.g., of an amide bond, carbamate bond, ether bond, or other linkage between the peptide and the therapeutic moiety.

The therapeutic moieties of the invention are typically linked to the peptides of the invention, either directly or indirectly (e.g., via a covalent bond, or a spacer or linker molecule). The attachment or linkage of the therapeutic moiety or cytotoxic moiety to the peptide moiety of the invention typically results in limiting the toxicity or function of the moiety while attached to the peptide. The moiety is then activated or available for use after being cleaved from the peptide. Therefore, the prodrugs of the invention are not generally toxic. For example, a cytotoxic moiety has an affect only when cleaved, e.g., in the presence of hepsin.

When a linker is used to attach the therapeutic moiety to the peptide portion of the prodrug, the linker is optionally cleaved from the peptide moiety along with the therapeutic moiety, or it remains behind. If the linker remains with the therapeutic moiety after cleavage by hepsin, it does not typically affect the function or toxicity of the therapeutic moiety.

In other embodiments, the linker or spacer group is self-cleaving. Self cleaving or self-immolative linkers are those designed to cleave or spontaneously eliminate from the therapeutic moiety after cleavage of the therapeutic moiety from the peptide. For information on self-cleaving linkers useful in prodrugs, see, for example, U.S. Pat. No. 6,265,540 B1, entitled “Tissue Specific Prodrug” by Isaacs et al., issued Jul. 24, 2001.

A “therapeutic moiety” of the invention is a compound, molecule, substituent, or the like, that relates to the treatment or prevention of a disease or disorder, e.g., to provide a cure, assist in a cure or partial cure, or reduce a symptom of the disease or disorder. For example, a cytotoxic moiety or an anti-metastatic moiety is used to treat cancer, e.g., by killing cancer cells or preventing their spread. In the present invention, therapeutic moieties are typically linked to the carboxyl terminus of the peptides of the invention, e.g., at P₁ or P₋₁. The therapeutic moiety or drug is optionally linked directly to the peptide or via a linker. Direct linkage typically involves an amide bond or an ester bond. When a linker is used, any type of linkage or bond known to those of skill in the art is optionally used.

A “cytotoxic moiety” is one that is toxic to cells, e.g., cancer cells. Such toxic moieties are used, e.g., to kill cancer cells. In the present invention, cytotoxic moieties are attached to hepsin substrates and targeted to the site of a cancer. The toxic moiety is released at the site of a cancer, e.g., at a tumor site, by the cleavage of the cytotoxic moiety from the prodrug, e.g., by hepsin, thereby killing the cells in the area. In this manner, the toxic moiety is preferably targeted to and released only at sites that are high in hepsin activity, as opposed to being released throughout the body to randomly kill cells, e.g., cancerous and non-cancerous. Therefore, the cytotoxic moiety is released in such a way as to reduce general toxicity to the body while killing cancerous cells as intended. Cytotoxic moieties of the invention include, but are not limited to, doxorubicin, daunorubicin, epirubicin, idarubicin, anthracycline, paclitaxel, camptothecin, mitomycin C, phenylenediamine mustard, and the like.

FIG. 2A shows a peptide substrate, e.g., PRLR, linked to doxorubicin to form a prodrug of the invention. Doxorubicin is released from the peptide only in the presence of hepsin, e.g., after cleavage. FIG. 2B depicts another peptide substrate PKLK, linked to doxorubicin through a self-immolative linker or spacer group consisting of the 4-aminobenzyl carbamate moiety. In the presence of hepsin, the peptide is cleaved from the linker at the C-terminus. The linker then spontaneously eliminates, resulting in the release of free (active) doxorubicin. FIG. 2C shows a further peptide substrate PKLK, linked to camptothecin through a self-immolative linker consisting of the 4-aminobenzyl ether moiety. In the presence of hepsin, the peptide is cleaved from the linker at the C-terminus. The linker then spontaneously eliminates, resulting in the release of free camptothecin.

In some embodiments, the prodrugs of the present invention comprise cell-modulating moieties, such as apoptosis-inducing or necrosis-inducing signal moieties, or anti-metastatic or antiproliferative signal moieties, e.g., that are cleaved from the prodrug at a cancerous site. A “cell modulating moiety” is any compound, reagent, molecule, or the like, that has an effect on the functioning of a cell. For example, a cell modulating moiety optionally cause the death of a cell, prevents the growth of a cell, or the like. As with the therapeutic moieties discussed above, the cell-modulating moieties typically have no effect until they have been cleaved from the prodrug to form an active drug, e.g., at the site of a cancerous tumor.

Necrosis or apoptosis are two mechanisms by which living cells typically die. Necrosis typically refers to cell death resulting from trauma, e.g., caused by an external force. Apoptosis, or programmed cell death, refers to an orderly sequence of responses to biochemical or physical signals that end in cell death. This is the body's mechanism for removal of unwanted or damaged cells. Cell death is normally a tightly-regulated process in which cells are constantly reacting to chemical signals from other cells or from their environment, e.g., instructing them to live or die. If signals instructing cells to live are lost, disease or death may result. For example, in degenerative diseases such as Alzheimer's disease, too many brain cells die inappropriately. In cancer, not enough cells die, resulting in uncontrolled growth. In some embodiments of the present invention, anti-cancer compounds are optionally used to induce apoptosis, e.g., selectively in precancerous and cancerous cells. An “apoptosis inducing signal moiety” is a compound, molecule, or substituent (e.g., a Fas ligand substituent) that induces apoptosis, e.g., by creating a cellular signal that causes the cell to begin programmed cell death or by inhibiting a signal instructing the cell to live. For example, a hepsin substrate or hepsin recognition site comprising a peptide of the invention and an apoptosis inducing signal moiety is optionally used to treat prostate cancer. The signal moiety is cleaved from the peptide and therefore becomes active when in the presence of hepsin, e.g., at the cancerous location, such as a prostate tumor. Similarly a “necrosis-inducing moiety” is a compound, molecule, substituent, or the like that is optionally linked to a hepsin substrate, which causes trauma or cell death, e.g., when cleaved from the hepsin substrate, e.g., at the site of tumor.

In other embodiments, a prodrug comprises an anti-metastatic moiety or an antiproliferative moiety. Metastasis is the spread of movement of cancer cells from a primary cancer site to another area of the body. During metastasis, tumor cells penetrate fibrous boundaries that normally separate one tissue from another. For example, tumor cells from colon cancer can invade the circulatory system and be carried to the liver where secondary tumors arise. Typically, metastasis occurs only after certain genes are turned on. These genes produce enzymes necessary for the cancer cells to penetrate other tissues and invade blood vessel walls. These enzymes and receptors for these enzymes provide putative targets for drugs that block metastasis. An “anti-metastatic moiety” or an antiproliferative moiety is one that prevents the spread of cancer cells or metastasis, e.g., by providing a signal that blocks the enzymes necessary for the spread of cancer cells. In the present invention, anti-metastatic and antiproliferative signals, e.g., angiostatin, endostatin, or matrix metalloproteinase (MMP) inhibitors, are typically attached to hepsin substrates, e.g., peptides. The signals are typically only released from the peptide or substrate after being cleaved by hepsin.

To form efficient and/or functional prodrugs, e.g., that result in the death of cancerous cells, additional moieties are also optionally attached to the substrates of the invention, e.g., to improve the solubility of the substrate, e.g., in water. For example, polysaccharides and/or starches are optionally attached to the peptides of the invention, e.g., at P₄ or P₋₁. Other groups optionally linked to the peptides of the invention include protecting groups, e.g., for protecting the peptides from degradation, e.g., by endopeptidases. For example, acetyl and succinyl are optionally used to cap the ends of the peptides of the invention, e.g. to prevent degradation. Pegylation (e.g., the attachment of PEG, or polyethylene glycol) is another common option. In other embodiments, various components, e.g., polyethylene glycol or a polysaccharide, are optionally added to the peptides of the invention, e.g., to shield or prevent a label or cytotoxic reagent form becoming active prior to cleavage by hepsin.

In another aspect, the present invention provides methods of treating cancer, e.g., prostate cancer, methods of inhibiting cancer cells, and methods of killing cancer cells, e.g., using the prodrugs of the invention. For example, the prodrugs of the invention are optionally administered to a subject, e.g., a mammalian subject, such as a human. Routes of administration include, but are not limited to, intravenous, intraperitoneal, intramuscular, subcutaneous, transdermal, or other methods known to those of skill in the art.

When administered to a subject, the prodrugs of the invention are typically provided in an aqueous or non-aqueous solution, suspension, or emulsion. Suitable solvents are known to those of skill in the art and include, but are not limited to, polyethylene glycol, ethyl oleate, water, saline, and the like. Preservatives, and other additives are also optionally included, e.g., antimicrobials.

For more information regarding anti-cancer prodrugs, e.g., enzymatically cleavable small molecules attached to therapeutics moieties, see, e.g., “Cathepsin B-Sensitive Dipeptide Prodrugs,” by Dubowchik et al. (1998) Bioorganic and Medicinal Chemistry Letters 8:3347-3352; “Synthesis and Biological Evaluation of Novel Prodrugs of Anthracyclines for Selective Activation by the Tumor-Associated Protease Plasmin,” by Groot et al. (1999) J. Med. Chem. 42:5277-5283; “Protease activated “Prodrugs” for Cancer Chemotherapy,” by Carl et al. (1980) Proc. Natl. Acad. Sci USA 77:2224-2228; and “A Peptide-Doxorubicin ‘Prodrug’ Activated by Prostate-Specific Antigen Selectively Kills Prostate Tumor Cells Positive for Prostate-Specific Antigen in Vivo,” by Jones et al. (2000) Nature Medicine 6:1248-1252.

Hepsin Substrate Based Diagnostics

In addition to prodrugs, the hepsin substrates of the present invention are also used as diagnostic reagents or components thereof. For example, a hepsin substrate of the invention is optionally linked to a fluorescent molecule, e.g., one that fluoresces only after cleavage from the substrate, to provide a diagnostic moiety that is used to detect the presence of hepsin or in high throughput screening of hepsin inhibitors.

A “diagnostic moiety” is a compound, molecule, substituent, or the like, that is used, e.g., to distinguish or identify, e.g., a certain disease, condition, or diagnosis. For example, the presence of hepsin is an example of a condition that a diagnostic of the invention is optionally used to identify. A diagnostic moiety of the invention is typically a label moiety that fluoresces upon cleavage from a hepsin substrate and allows the detection of the cleavage event, e.g., that is used to detect the presence of hepsin, e.g., in a tumor cell.

A “label moiety” is any detectable compound, molecule, or the like. The labels in the present invention typically provide for detection of hepsin. For example, the labels used are typically attached to a hepsin substrate of the invention. Typically, the labels of the present invention do not become detectable until after a cleavage event has occurred, e.g., cleaving the label from a hepsin substrate. A label is detectable by any of a number of means, such as fluorescence, phosphorescence, absorbance, luminescence, chemiluminescence, radioactivity, colorimetry, magnetic resonance, or the like.

Label moieties of the invention include, but are not limited to, absorbent, fluorescent, or luminescent label moieties. Exemplary label moieties include, but are not limited to fluorophores, coumarin moieties, such as 7-amino-4-carbamoylcoumarin, 7-amino-3-carbamoyl-4-methylcoumarin, or 7-amino-4-methylcoumarin, or rhodamine moieties. Typically, a label moiety exhibits significantly less absorbance, fluorescence or luminescence when attached to the hepsin-cleavable molecule than when released from the hepsin-cleavable molecule.

For example, a fluorophore emits light when it is exposed to the wavelength of light at which it fluoresces. The emitted light is detected. In the present invention, fluorophores that do not fluoresce until separated from the attached peptide are typically used. Therefore, a hepsin substrate with an attached fluorophore does not fluoresce or provide a signal until the substrate is cleaved by hepsin, thereby releasing the fluorophore. In this manner, the presence of hepsin is easily detected using the substrates of the invention. In addition, a library of putative substrates is easily screened against hepsin to determine which putative substrates are actually cleaved by hepsin. Fluorophores of interest include, but are not limited to, fluorescein, fluorescein analogs, BODIPY-fluorescein, arginine, rhodamine-B, rhodamine-A, rhodamine derivatives, green fluorescent protein (GFP), and the like. For further information on fluorescent label moieties and fluorescence techniques, see, e.g., Handbook of Fluorescent Probes and Research Chemicals, by Richard P. Haugland, Sixth Edition, Molecular Probes, (1996).

An exemplary label moiety that does not fluoresce until cleaved from the substrate is a coumarin moiety. A “coumarin moiety” is a compound or molecule comprising a coumarin compound. Coumarin compounds of interest in the present invention include, but are not limited to, 7-amino-4-carbamoylmethylcoumarin (“acc”), 7-amino-4-methylcoumarin (“amc”), 7-methoxy-4-carbamoylmethylcoumarin, and 7-dimethylamino-4-carbamoylmethylcoumarin, and the like. An exemplary hepsin substrate linked to a coumarin moiety is provided in FIG. 1. P₄-P₁ are optionally any amino acid sequence, e.g., as provided above. Many other coumarin compounds are available, e.g., either commercially (see, e.g., Sigma and Molecular Probes catalogs) or using various synthetic protocols known to those of skill in the art. The synthesis of an exemplary coumarin compound of interest is provided in International Patent Application PCT/US02/27357 by Backes et al, supra.

For basic strategies for preparation of and use of coumarin-based substrates and coumarin libraries, see, e.g., Zimmerman et al. (1977) Analytical Biochemistry 78:47-51; Lee et al. (1999) Bioorganic and Medicinal Chemistry Letters 9:1667-72; Rano et al., supra; Schechter and Berger (1968) Biochemical and Biophysical Chemistry Communications 27:157-162; Backes et al. (2000) Nature Biotechnology 18:187-193; Harris et al. (2000) “Rapid and general profiling of protease specificity by using combinatorial fluorogenic substrate libraries” Proc. Natl. Acad. Sci USA 97:7754-7759; and Smith et al. (1980) Thrombosis Res. 17:393-402. See, also, PCT/US02/27357 by Backes et al, supra.

In other embodiments, quantum dots are optionally used as diagnostic moieties. Nanocrystals, e.g., semiconductor nanocrystals or quantum dots such as cadmium selenide and cadmium sulfide, are optionally used as fluorescent probes. Quantum dots typically emit light in multiple colors, which allows them to be used to label and detect several compounds or samples at once. See, e.g., Bruchez et al. “Semiconductor Nanocrystals as Fluorescent Biological Labels,” Science 281:2013-2016 (1998). Quantum dot probes are available, e.g., from Quantum Dot Corporation (Hayward, California).

In the present invention, a quantum dot is optionally linked to or associated with a hepsin substrate or a putative hepsin substrate and used to detect the substrate, e.g., after cleavage by hepsin. In some embodiments, the label moiety optionally comprises a first quantum dot attached to a hepsin cleavable molecule on one side of the hepsin cleavage site and a second quantum dot attached to the molecule on the opposite side of the hepsin cleavage site. Typically, the first and second quantum dots emit signals of different wavelengths upon illumination. For example, a quantum dot is optionally linked to a prime side of a peptide substrate as described above, e.g., using standard chemistry techniques, and a differently colored quantum dot is linked to the non-prime side of the substrate. Detection of the quantum dots allows detection of a cleavage event when the prime and non-prime sides are cleaved from each other, e.g., by hepsin.

Alternatively, electroactive species, useful for electrochemical detection, or chemiluminescent moieties, useful for chemiluminescent detection, are incorporated into the hepsin substrates or putative substrates of the invention. UV absorption is also an optional detection method, for which UV absorbers are optionally used. Phosphorescent, colorimetric, e.g., dyes, and radioactive labels are also optionally added to the attached to the hepsin substrates of the invention, e.g., using techniques well known to those of skill in the art.

Labels as described above are typically linked to the substrates or putative substrates of the invention using techniques well known to those of skill in the art. For example, the label or diagnostic moiety is typically linked to P₁ as shown below:

P₄P₃P₂P₁X

wherein X comprises the label moiety. Alternatively, the label moiety is linked to the prime side of a hepsin substrate or to P₄. In some embodiments, the label moiety comprises two labels, such as two quantum dots. One label is attached to the prime side of the substrate and the other label is attached to the non-prime side of the substrate, as shown below:

′X₁P₄P₃P₂P₁P₁′P₂′P₃′P₄′X₁′

wherein X₁ and X₁′ each comprise a label moiety, such as quantum dot or a member of a FRET pair. In other embodiments, the label moiety is optionally attached to any of the substrate moieties, e.g., P₄-P₁, or P₁′-P₄′.

The diagnostics, e.g., the hepsin substrates linked to one or more label moiety, are then optionally used in high throughput screening applications, e.g., screening a library of putative substrates for hepsin substrates, or identifying hepsin inhibitors or activators.

The present invention also provides methods of labeling a cell using the labeled hepsin-cleavable molecules of the present invention. The labeling method include contacting the cell with a hepsin-cleavable molecule that comprises a hepsin cleavage site, wherein the hepsin-cleavable molecule comprises the structure P₄P₃P₂P₁X, wherein the hepsin cleavage site is between P₁ and X; and wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a label moiety. A variety of labels can be incorporated into the hepsin-cleavable molecules of the present invention, including, but not limited to, a coumarin moiety and members of a donor-acceptor FRET pair, as described herein. Optionally, the cell comprises a prostate tissue cell.

In a further aspect, the present invention provides methods of screening an individual for increased hepsin activity or expression. The methods include the steps of a) obtaining a cell or tissue sample from the individual; b) contacting the cell or tissue sample with one or more hepsin-cleavable molecules that comprise a hepsin cleavage site (for example, a hepsin substrate molecule P₄P₃P₂P₁X wherein the hepsin cleavage site is between P₁ and X; and wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and wherein X comprises a label moiety); and c) detecting a release of the label moiety from the hepsin cleavable molecule, thereby screening the individual for increased hepsin activity or expression. The hepsin activity is optionally diagnostic of a disease, e.g., a cellular metabolic state in which the hepsin expression or activity is altered. Optionally, the level of detected label is compared to a control or standard level of hepsin activity, thereby determining whether the hepsin activity or expression is increased. These methods performed on a cell from prostate tissue can be used as an indication or diagnostic of prostate cancer.

Hepsin Inhibitors

Inhibitors are typically compounds or molecules that negatively affect the ability of an enzyme to catalyze a reaction. For example, an inhibitor inhibits or curbs enzyme activity. Pepstatin is an example of a protease inhibitor because it inhibits the activity of carboxyl proteases. A “hepsin inhibitor” is a protease inhibitor that inhibits, curbs, or decreases the activity of hepsin.

In one aspect, the present invention provides hepsin inhibitors. The inhibitors typically comprise a hepsin recognition site such as a peptide sequence as described above. The peptide sequence is typically linked to an inhibitory moiety, Z. For example, a typical inhibitor is shown below:

P₄P₃P₂P₁Z

wherein P₁-P₄ are defined as described above, and Z comprises a component that is capable of inhibiting hepsin activity when associated with the hepsin protease, such as a transition state analog, a mechanism-based inhibitor, an electron withdrawing group, a chemical modifier, or the like.

Serine proteases typically have a similar active site geometry, such that hydrolysis of the substrate bond proceeds via the same mechanism of action. The first step in the reaction is the formation of an acyl-enzyme intermediate between the substrate and a conserved serine residue in the active site (hence the classification as a “serine protease”). The peptide bond is cleaved during formation of this covalent intermediate, which proceeds via a (negatively charged) tetrahedral transition state intermediate. Deacylation occurs during the second step of the mechanism of action, at which point the acyl-enzyme intermediate is hydrolyzed by a water molecule, the remaining portion of the substrate peptide is released, and the hydroxyl group of the serine residue is restored. The deacylation process also involves the formation of a tetrahedral transition state intermediate. As such, transition state analog compounds which mimic the structure of either of the tetrahedral intermediates can be employed as inhibitors of the serine protease.

Furthermore, chemical constituents which covalently modify or otherwise interact with the active site of the hepsin molecule can also be used as inhibitor moieties in the present invention. In some embodiments of the present invention, cleavage of the hepsin inhibitor molecule irreversibly deactivates the hepsin protease (e.g., a suicide inhibitor). In other embodiment, the inhibitor moiety need not be released from the hepsin inhibitor molecule to function as an inhibitor (e.g., an inhibitory affinity label). Optionally, the inhibitor moiety is activated upon release from the hepsin recognition site, and functions to either inhibit a single hepsin molecule or to catalyze the inhibition of a number of hepsin molecules. Mechanisms of serine protease inhibition are further described in Fersht (1985) Enzyme Structure and Mechanism (W. H. Freeman and Company, New York).

Contacting hepsin with the hepsin inhibitors of the invention results in complete or partial inactivation of hepsin. In some inhibitor embodiments, P₄ comprises acetyl lysine. The transition state analog, mechanism-based moiety, or electron withdrawing moiety optionally comprises a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide, or the like.

In one embodiment, hepsin specific aldehyde inhibitors are provided. For example, a peptide with a C-terminal aldehyde is provided. The peptide sequence is typically based on the substrate specificity of hepsin. For example, an inhibitor is optionally based on one or more of the hepsin substrates identified above, e.g., KRLR, KQLR, PQLR, RQLR, RRLR, PRLR, PKLK, PKLR, or PRLK. In one embodiment, a hepsin inhibitor comprises Acetyl-P₄-P₃-P₂-P₁-aldehylde, wherein P₄-P₁ comprise a non-prime substrate sequence as provided above. An example structure, e.g., N-acetyl-K-R-L-R-al, is illustrated below by Formula I,

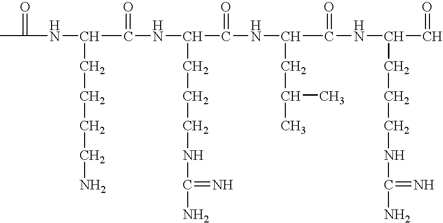

Formula I provides a hepsin inhibitor based on the hepsin substrate specificity profiles determined as provided herein.

The aldehyde inhibitor provided by Formula I is optionally prepared using semicarbazone methodology. See, e.g., Dagino and Webb (1994) Tetrahedron Letters 35: 2125-2128. Dagino and Webb describe a method of making peptide aldehydes which involves using a diphenylmethyl semicarbazone group to provide a synthetic intermediate. For example, a protected diphenylmethyl semicarbazide derivative is synthesized, e.g., using techniques known to those of skill in the art. The semicarbazide is reacted to provide a protected argininal derivative, which is converted to a free amine, to which a desired peptide is linked, e.g., using standard peptide coupling techniques. The fully protected peptide aldehydes produced in this manner are optionally purified, e.g., using silica chromatography, and deprotected, e.g., by hydrogenation in acidic aqueous methanol.

The present invention also provides methods of reducing a hepsin activity in a cell. The methods involve contacting the cell with a hepsin inhibitor molecule containing a hepsin recognition site. Typically, the hepsin inhibitor molecule comprises a compound comprising the structure P₄P₃P₂P₁X, wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and wherein X comprises an inhibitory moiety, such as a transition state analog, a mechanism-based inhibitor, or an electron withdrawing group. Exemplary inhibitory moieties include, but are not limited to, a C-terminal aldehyde, a boronate, a phosphonate, an α-ketoamide, a chloro methyl ketone, a sulfonyl chloride, ethyl propenoate, vinyl amide, vinyl sulfone, or vinyl sulfonamide.

Therapeutic and Phrohylactic Treatment Methods

In one aspect, the present method provides methods of killing a cell, the methods comprising contacting the cell with a hepsin-cleavable molecule that comprises a hepsin cleavage site, wherein the hepsin-cleavable molecule comprises P₄P₃P₂P₁X, wherein the hepsin cleavage site is between P₁ and X; and wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a cytotoxic moiety. Exemplary cytotoxic moieties for use in the methods of the present invention include, but are not limited to, doxorubicin, daunorubicin, epirubicin, idarubicin, anthracycline, paclitaxel, camptothecin, mitomycin C, phenylenediamine mustard, one or more bacterial toxins, or a combination thereof. Optionally, the cytotoxic moiety further includes a linker moiety for attachment to the P₁ substituent (e.g., a peptide sequence, such as a hepsin cleavable molecule having the formula P₄P₃P₂P₁P₁′P₂′P₃′P₄′X).

Any of a variety of cells expressing hepsin activity can be targeted and killed by the methods of the present invention, such as mammalian cells (including, e.g., a human, primate, mouse, pig, cow, goat, rabbit, rat, guinea pig, hamster, horse, sheep) and cells from non-mammalian vertebrates such as bird, fish, amphibians and invertebrates. In a preferred embodiment, the cell targeted by the methods of the present invention comprise cancer cell or a cell overexpressing a hepsin activity. In one embodiment of the methods, contacting the cell with a hepsin-cleavable molecule is performed in vitro, such as performing an in vitro assay on cultured cells. In an alternate embodiment, contacting the cell comprises administering the hepsin cleavable molecule to the cell in vivo. Optionally, the one or more cells are present in a subject, such that the hepsin substrates, inhibitors or prodrugs are administered in vivo.

The methods of the present invention also encompasses methods of therapeutically or prophylactically treating a disease or disorder, by contacting or administering to one or more cells one or more hepsin substrates, inhibitors or prodrugs of the present invention (or compositions comprising a pharmaceutically acceptable excipient and one or more such hepsin substrates, inhibitors or prodrugs). In these in vivo methods, one or more cells of the subject, or a population of cells of interest, are contacted directly or indirectly with an amount of a hepsin substrate, inhibitor or prodrug composition of the present invention effective in prophylactically or therapeutically treating the disease, disorder, or other condition (e.g., a prostate cancer, or an overexpression of hepsin activity). In direct contact/administration formats, the composition is typically administered or transferred directly to the cells to be treated or to the tissue site of interest (e.g., the prostate or other hepsin-expressing tissue). In in vivo indirect contact/administration formats, the composition is typically administered or transferred indirectly to the cells to be treated or to the tissue site of interest (e.g., by the circulatory system or the lymph system). Any of a variety of formats can be used to administer the compositions of the present invention (optionally along with one or more buffers and/or pharmaceutically-acceptable excipients), including topical administration, transdermal administration, oral delivery, injection (e.g., by using a needle or syringe), placement within a cavity of the body (e.g., by catheter or during surgery), and the like. Pharmaceutically-acceptable excipients for use in the present invention include, but are not limited to, saline, buffered saline, dextrose, water, glycerol, ethanol, conventional nontoxic binders, disintegrants, flavorings, and carriers (e.g., pharmaceutical grades of mannitol, lactose, starch, magnesium stearate, sodium saccharine, talcum, cellulose, glucose, sucrose, magnesium, carbonate, and the like) and combinations thereof. The formulation is made to suit the mode of administration. Exemplary excipients and methods of formulation are provided, for example, in Remington's Pharmaceutical Science, 17th ed. (Mack Publishing Company, Easton, Pa., 1985).

Therapeutic compositions comprising one or more hepsin substrates, inhibitors or prodrugs of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Kits

In an additional aspect, the present invention provides kits embodying the compositions and/or methods provided herein. Kits of the invention optionally comprise one or more of the following: (1) a composition or library of compositions as described herein; (2) instructions for practicing the methods described herein, and/or for using the compositions described herein; (3) a hepsin protease or an expression vector for generation of the hepsin protease; (4) a container for holding components or compositions, and, (5) packaging materials.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Determination of Non-Prime Side Substrate Specificity

The non-prime side specificity of recombinantly-expressed hepsin was determined using positional scanning combinatorial tetrapeptide libraries with a C-terminal latent fluorophore as provided in FIG. 1. Two different libraries were used.

The first library was a 1-position fixed library in which one position is known and the other three positions contain an equal molar mixture of 19 amino acids. Cysteine is not typically included and norleucine is typically substituted for methionine, providing a total of 6,859 substrates per well. The resulting profile for hepsin specificity is shown in FIG. 3, in which the x-axis represents the “fixed” position in the tetrapeptide sequence, the y-axis represents the amino acid positioned at the fixed position, and the z-axis represents the hydrolysis rate in relative fluorescence units per second (RFU/s). The resulting profile demonstrates that hepsin prefers to cleave after basic amino acids, e.g., arginine and lysine (low intensity signal in this graph).

The second library used to profile hepsin substrate specificity is the two position fixed library, in which two positions are held constant in a tetrapeptide sequence and the other two positions contain an equal molar mixture of 19 amino acids as described above, e.g., for a total of 361 substrates per well. The substrate specificity of hepsin obtained from this library is shown in FIG. 4, Panels A-C. The amino acids held constant in each position are identified on the x- and y-axes and the shade of the square represents the rate of hydrolysis in RFU/s.

As in the 1-position fixed library, results from the 2-position fixed library also indicate that hepsin prefers to cleave after arginine and lysine (e.g., in the P₁ position). Preference in the P₂ position is for large aliphatic amino acids, e.g., valine, leucine, isoleucine, and methionine as well as basic amino acids, e.g., arginine, lysine, and histidine, and polar amino acids such as asparagine and threonine. The P₃ position prefers basic amino acids as well as the polar amino acids, glutamine, serine, and threonine. The P₄ position also prefers basic amino acids, but can also accommodate the majority of hydrophobic and aliphatic amino acids.

Using the substrate specificity profile as described above, several tetrapeptides were designed, synthesized, and tested for hepsin activity. For example, the following tetrapeptides were constructed: KRLR, KALR, and PRLR (all listed N-terminus to C-terminus, i.e., P₄-P₁, wherein Pₙ is used to represent any substrate moiety, e.g., amino acid, as opposed to the amino acid proline which is typically represented by the letter “P” without a subscript. Alternatively, the non-prime and prime side substrate moieties are represented by Xₙ and X₋ₙ.) Typically, a N-terminus acetyl protecting group is used as well as a coumarin moiety at the C-terminus.

The substrates were used to monitor hepsin activity in cell lines and tissues that express hepsin. For example, hepsin-like activity in relation to these substrates has been observed in the membrane fraction of several prostate cancer cell lines.

### Example 2

### Determination of Prime Side Substrate Specificity

A donor quencher library, e.g., as described above and in International Patent Application PCT/US02/27357 by Backes et al, supra, was used to determine prime side substrate specificity. The donor was a methoxy coumarin at the C-terminus of the peptide substrate and the quencher was a dinitrotyrosine at the N-terminus of the substrate.

The non-prime side of the substrate in the substrate library was kept constant at P₄-Arg, P₃-Lys, P₂-Leu, P₁-Arg. Other substrate sequences, e.g., as described above, are also optionally used. The prime-side four amino acid positions were randomized as all 20 natural amino acids, with the exception that norleucine replaced methionine and cysteine was excluded. The results for each of the positions are shown in the histograms of FIG. 9, panels A-D, with the y-axis representing relative fluorescence units per second and the x-axis representing the amino acid held constant in the prime side site (P₁′, P₂′, P₃′ or P₄′) of the substrate.

### Example 3

### Expression of Hepsin in Insect Cells

Expression in insect cells produces reagent amounts of soluble and active hepsin, e.g., >1 mg/L. The constructs and methods are described below.

For purposes of high-throughput screening of compound collections and crystallization, a soluble form of hepsin (e.g., without the transmembrane domain) is preferred. The honeybee melittin secretion signal sequence (Mel) was appended to the N-terminus of a hepsin sequence by two rounds of PCR extension. Two fragments of hepsin were selected for expression, Hep136 (covering the prodomain and the catalytic domain; amino acids 46-417; see FIG. 5, bold amino acids) and HepCat (covering only the catalytic domain; amino acids 163-417; see FIG. 5, underlined amino acids). To facilitate the downstream purification, a 6×His tag was appended to the C-terminus of the hepsin fragments. All PCR constructs were inserted into a pFastBac1 (Invitrogen Corporation, Carlsbad, Calif.) vector using EcoR I and Not I sites.

FIG. 6 provides a diagram of the modified pFastBac1 vector used for hepsin expression. Unique restriction sites Mlu I and Pvu II were also introduced, e.g., to make this vector a general tool for baculoviral expression of secreted proteins. Using the Mel modified pFastBac1 vector, the following four plasmids we made and amplified following the manufacturer's protocol:

Mel-Hep136

Mel-HepCat

Mel-Hep136-6His

Mel-HepCat-6His

SP9 cells were infected with recombinant virus at a MOI of 5-10 and the activity of hepsin in the supernatant was monitored by the hydrolysis of a fluorogenic peptide, KRLR-ACC. Activities were only observed in the supernatant of two Hep136 constructs but not in the supernatant of two HepCat constructs, suggesting the prodomain is required for hepsin production or activity. To determine the optimal expression time, activity was monitored for 72 hours after infection. Optimal activity/expression was observed at 48 hours after infection, at which time the supernatant was collected, cleared by centrifugation, and stored at 4C.

Native Hep136 was purified as follows: one volume of 100 mM Tris, pH 7.8, 3.4 M (NH₄)₂SO₄ was slowly added with stirring to the supernatant. The precipitate were cleared by centrifugation and the supernatant was applied to a phenyl sepharose column, washed with 50 mM Tris, pH 7.4.0, 0.8 M (NH₄)₂SO₄ and eluted with 50 mM Tris, pH 7.4, 0.02% Tween-20.

His-tagged Hep136 was purified as follows: the pH of supernatant from SF9 culture was adjusted to 8.0 with 0.5N NaOH. After centrifugation, the supernatant was concentrated and dialyzed extensively against 50 mM Hepes, pH 7.4, 200 mM NaCl and 5 mM CaCl₂. The supernatant was then applied to Ni-NTA column, washed with 2 mM imidazole and eluted with 250 mM imidazole.

To ensure that the 6×His tag on the C-terminus does not significantly change the substrate specificity of hepsin, Hep136 and His-tagged Hep136 were profiled in the two-position fixed substrate library. The results are provided in FIGS. 7 and 8. The profiles are consistent with that observed from the refolded hepsin produced in E. coli, with major activity observed in P₁-Arg and P₁-Lys. Furthermore, comparison shows that the C-terminal histidine tag does not significantly affect the substrate specificity of hepsin.

The activity of hepsin expressed and purified as described above was tested using the following substrates, which were synthesized based on the substrate specificity profile from the positional scanning libraries described above: Acetyl-Pro-Arg-Leu-Arg-ACC/ACMC and Acetyl-Lys-Arg-Leu-Arg-ACC/ACMC. Both sequences are efficiently cleaved by recombinant hepsin expressed as described above, e.g., with and without a histidine tag.

### Example 4

### Identification of Physiological Hepsin Substrate

Based on non-prime specificity determinants of hepsin, e.g., determined from a coumarin-based substrate library and prime-side specificity determinants of hepsin (e.g., determined from a donor-quencher library), several possible physiological hepsin substrates are optionally identified. Among these is pro-urokinase plasminogen (pro-uPA), an activator with a cleavage sequence comprising Pro-Arg-Phe-Lys-Ile-Ile-Gly-Gly. Cleavage typically occurs between Lys and Ile. Cleavage of pro-uPA at this site leads to activation of its proteolytic activity. Active uPA can then proceed to activate plasminogen to generate the active protease plasmin. Plasmin has been shown to activate pro-matrix metalloproteinases (pro-MMPs) whose activity can lead to extracellular matrix remodeling, primary tumor growth, and/or metastasis.

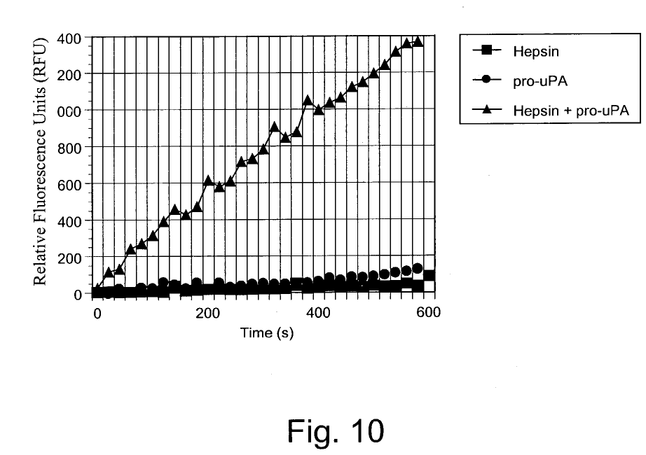

To test whether hepsin is capable of activating pro-uPA (and thereby potentially initiating this proteolytic cascade), uPA was screened through a coumarin-based substrate library and a substrate was designed to monitor uPA proteolytic activity. The substrate designed to monitor uPA activity is Acetyl-Gly-Thr-Ala-Arg-[7-amino-4-carbomoylcoumarin] (GTAR-acc). 2 μM of pro-uPA was incubated with 1 nM of recombinant hepsin and 100 μM of GTAR-acc. At the concentrations of enzyme and substrate used, hepsin only marginally cleaves GTAR-acc as shown in FIG. 10 (squares). Also shown in FIG. 10 is that hepsin can indeed accelerate the activation of pro-uPA, e.g., at concentrations used hepsin shows a 10-fold activation of pro-uPA over background auto-activation (compare triangles to circles).

The discussion above is generally applicable to the aspects and embodiments of the invention described in the claims. Moreover, modifications can be made to the methods and compositions described herein without departing from the spirit and scope of the invention as claimed, and the invention can be put to a number of different uses including the following:

Use of a hepsin substrate, prodrug, inhibitor or diagnostic compound for analysis of a hepsin activity.

Use of a hepsin substrate, prodrug, inhibitor or diagnostic compound for labeling or killing a cell.

Use of a hepsin substrate, prodrug, inhibitor or diagnostic compound for diagnosing a disease condition involving hepsin or alterations in hepsin activity.

Use of a hepsin substrate, prodrug, inhibitor, diagnostic compound, or other hepsin-cleavable molecule as described herein for screening a library of compounds for a modulator of hepsin activity.

Use of an assay or method utilizing a hepsin substrate, prodrug, inhibitor, diagnostic compound or expression vector as described herein, e.g., for practicing any method or assay set forth herein.

Use of a hepsin expression vector, for performing any of the methods and assays set forth herein.

Use of kits comprising any a hepsin substrate, prodrug, inhibitor, diagnostic compound or expression vector, e.g., for practicing any method or assay set forth herein, or for facilitating practice of any method or use of any composition set forth herein.

While the foregoing invention has been described in some detail for purposes of clarity and understanding, it will be clear to one skilled in the art from a reading of this disclosure that various changes in form and detail can be made without departing from the true scope of the invention. For example, all the techniques and compositions described above may be used in various combinations, and other uses for the present invention are also contemplated. All publications, patents, patent applications, and/or other documents cited in this application are incorporated by reference in their entirety for all purposes to the same extent as if each individual publication, patent, patent application, or other document were individually indicated to be incorporated by reference for all purposes.

What is claimed is:

1. A hepsin-cleavable molecule that comprises a hepsin cleavage site, wherein the hepsin-cleavable molecule comprises:
P₄P₃P₂P₁X
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine;
P₄ is arginine, lysine, proline, valine, leucine, isoleucine, methionine, norleucine, alanine, glycine, tryptophan, phenylalanine, or tyrosine; and
X comprises one or more of a cell modulating moiety, a label moiety, a polypeptide comprising 1 to 25 amino acids, or a polypeptide that is not attached to P₄P₃P₂P₁ in a naturally occurring protein; and
wherein the hepsin cleavage site is between P₁ and X.

2. The hepsin-cleavable molecule of claim 1, wherein P4 is selected from the group consisting of arginine, lysine, proline, valine, leucine, and alanine.

3. The hepsin-cleavable molecule of claim 1, wherein P₄P₃P₂P₁ comprises KRLR.

4. The hepsin-cleavable molecule of claim 1, wherein P₄P₃P₂P₁ comprises KQLR, PQLR, RQLR, or RRLR.

5. The hepsin-cleavable molecule of claim 1, wherein P₄P₃P₂P₁ comprises PRLR.

6. The hepsin-cleavable molecule of claim 1, wherein P₄P₃P₂P₁ comprises PKLK, PKLR, or PRLK.

7. The hepsin-cleavable molecule of claim 1, wherein X comprises:
P₁′P₂′P₃′P₄′
wherein:
P₁′ is methionine, norleucine, leucine, isoleucine, valine, alanine, tyrosine, or threonine;
P₂′ is alanine, phenylalanine, tyrosine, threonine, histidine;
P₃′ is arginine, lysine, histidine, glutamine, serine, threonine, tyrosine, tryptophan, glycine, leucine or methionine, and
P₄′ is aspartic acid, glycine, proline, valine, or methionine.

8. The hepsin-cleavable molecule of claim 1, wherein X comprises a cell modulating moiety selected from the group consisting of a cytotoxic moiety, an antiproliferative moiety, an anti-metastatic moiety, an apoptosis-inducing moiety, and a necrosis-inducing moiety.

9. The hepsin-cleavable molecule of claim 8, wherein the cell modulating moiety is a cytotoxic moiety that comprises doxorubicin, daunorubicin, epirubicin, idarubicin, anthracycline, paclitaxel, mitomycin C, or phenylenediamine mustard.

10. The hepsin-cleavable molecule of claim 8, wherein the cytotoxic moiety comprises a bacterial toxin.

11. The hepsin-cleavable molecule of claim 1, wherein the cell modulating moiety is inactive until cleaved from the hepsin-cleavable molecule by hepsin.

12. The hepsin-cleavable molecule of claim 1, wherein the label moiety comprises an absorbent, fluorescent or luminescent label moiety.

13. The hepsin-cleavable molecule of claim 12, wherein the label moiety exhibits significantly less absorbance, fluorescence or luminescence when attached to the hepsin-cleavable molecule than when released from the hepsin-cleavable molecule.

14. The hepsin-cleavable molecule of claim 12, wherein the label moiety comprises a fluorophore, a coumarin moiety, or a rhodamine moiety.

15. The hepsin-cleavable molecule of claim 14, wherein the coumarin moiety comprises 7-amino-4-carbamoylcoumarin, 7-amino-3-carbamoyl-4-methylcoumarin, or 7-amino-4-methylcoumarin.

16. The hepsin-cleavable molecule of claim 12, wherein the hepsin-cleavable molecule comprises a first member of a fluorescence resonance transfer energy pair attached to the molecule on one side of the hepsin cleavage site and a second member of the fluorescence resonance transfer energy pair attached to the molecule on the opposite side of the hepsin cleavage site.

17. The hepsin-cleavable molecule of claim 16, wherein the fluorescence resonance transfer energy pair comprises amino benzoic acid and nitro-tyrosine; 7-methoxy-3-carbamoyl-4-methylcoumarin and dinitrophenol; or 7-dimethylamino-3-carbamoyl-4-methylcoumarin and dabsyl.

18. The hepsin-cleavable molecule of claim 12, wherein the hepsin-cleavable molecule comprises a first quantum dot attached to the molecule on one side of the hepsin cleavage site and a second quantum dot attached to the molecule on the opposite side of the hepsin cleavage site, wherein the first and second quantum dots emit signals of different wavelengths upon illumination.

19. An anti-cancer prodrug, which prodrug comprises a peptide sequence and a cytotoxic moiety, wherein the peptide sequence comprises:
P₄P₃P₂P₁
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine; and
P₄ is arginine, lysine, proline, valine, leucine, isoleucine, methionine, norleucine, alanine, glycine, tryptophan, phenylalanine, or tyrosine; and
wherein the cytotoxic moiety is attached to the peptide sequence and is inactive until the peptide sequence is cleaved by hepsin.

20. The prodrug of claim 19, wherein P₄P₃P₂P₁ comprises KRLR.

21. The prodrug of claim 19, wherein P₄P₃P₂P₁ comprises KQLR, PQLR, RQLR, or RRLR.

22. The prodrug of claim 19, wherein P₄P₃P₂P₁ comprises PRLR.

23. The prodrug of claim 19, wherein P₄P₃P₂P₁ comprises PKLK, PKLR, or PRLK.

24. The prodrug of claim 19, wherein the cytotoxic moiety comprises doxorubicin, daunorubicin, epirubicin, idarubicin, anthracycline, paclitaxel, mitomycin C, or phenylenediamine mustard.

25. The prodrug of claim 19, wherein the prodrug further comprises a polysaccharide, a saccharide, or polyethylene glycol.

26. The prodrug of claim 19, wherein the peptide sequence further comprises:
P₁′P₂′P₃′P₄′
wherein:
P₁′ is attached to P₁ and is methionine, norleucine, leucine, isoleucine, valine, alanine, tyrosine, or threonine;
P₂′ is alanine, phenylalanine, tyrosine, threonine, histidine;
P₃′ is arginine, lysine, histidine, glutamine, serine, threonine, tyrosine, tryptophan, glycine, leucine or methionine; and
P₄′ is aspartic acid, glycine, proline, valine, methionine.

27. A hepsin-cleavable peptide that comprises fewer than 25 amino acids, the peptide comprising:
P₄P₃P₂P₁
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine; and
P₄ is arginine, lysine, proline, valine, leucine, isoleucine, methionine, norleucine, alanine, glycine, tryptophan, phenylalanine, or tyrosine; and
one or more amino acids attached to either or both of P₁ and P₄.

28. The hepsin-cleavable peptide of claim 27, wherein P₁ is arginine, P₂ is leucine, P₃ is arginine or asparagine, and P₄ is lysine or proline.

29. The hepsin-cleavable peptide of claim 27, the peptide further comprising 1 to 20 amino acids linked to P₄.

30. The hepsin-cleavable peptide of claim 27, the peptide further comprising 1 to 20 amino acids linked to P₁.

31. The hepsin-cleavable peptide of claim 27, the peptide further comprising:
P₁′P₂′P₃′P₄′
wherein:
P₁′ is attached to P₁ and is methionine, norleucine, leucine, isoleucine, valine, alanine, tyrosine, or threonine;
P₂′ is alanine, phenylalanine, tyrosine, threonine, histidine;
P₃′ is arginine, lysine, histidine, glutamine, serine, threonine, tyrosine, tryptophan, glycine, leucine or methionine; and
P₄′ is aspartic acid, glycine, proline, valine, methionine.

32. A hepsin-cleavable peptide that comprises:
P₄P₃P₂P₁
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine; and
P₄ is arginine, lysine, proline, valine, leucine, isoleucine, methionine, norleucine, alanine, glycine, tryptophan, phenylalanine, or tyrosine; and
one or more molecules attached to either or both of P₁ and P₄ that are not attached to a polypeptide having a sequence P₄P₃P₂P₁ in a naturally occurring protein.

33. The hepsin-cleavable peptide of claim 32, wherein P₁ is arginine, P₂ is leucine, P₃ is arginine or asparagine, and P₄ is lysine or proline.

34. The hepsin-cleavable peptide of claim 32, the peptide further comprising:
P₁′P₂′P₃′P₄′
wherein:
P₁′ is attached to P₁ and is methionine, norleucine, leucine, isoleucine, valine, alanine, tyrosine, or threonine;
P₂′ is alanine, phenylalanine, tyrosine, threonine, histidine;
P₃′ is arginine, lysine, histidine, glutamine, serine, threonine, tyrosine, tryptophan, glycine, leucine or methionine; and
P₄′ is aspartic acid, glycine, proline, valine, methionine.

35. The hepsin-cleavable peptide of claim 32, further comprising one or more peptides, polyalcohols, biotin, or crosslinking agents coupled to the hepsin-cleavable peptide.

36. The hepsin-cleavable peptide of claim 35, wherein the polyalcohol comprises polyethylene glycol.

37. A library of putative hepsin substrates, wherein each member of the library comprises a putative hepsin cleavage site, wherein:
(a) the putative hepsin cleavage site comprises one or more non-prime positions and one or more prime positions, wherein the prime positions and the non-prime positions flank the putative hepsin cleavage site;
(b) the one or more non-prime positions are occupied by one or more preselected substrate moieties, which preselected substrate moieties are preselected to allow cleavage of the putative substrate at the putative cleavage site; and,
(c) the one or more prime positions are occupied by one or more substrate moieties, which substrate moieties vary among the members of the library of putative hepsin substrates.

38. The library of claim 37, wherein the preselected substrate moieties comprise a peptide sequence, which peptide sequence comprises:
P₄P₃P₂P₁
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine; and
P₄ is arginine, lysine, proline, valine, leucine, isoleucine, methionine, norleucine, alanine, glycine, tryptophan, phenylalanine, or tyrosine; and
one or more amino acids attached to either or both of P₁ and P₄.

39. The library of claim 38, wherein P₄P₃P₂P₁ comprises KRLR.

40. The library of claim 38, wherein P₄P₃P₂P₁ comprises KQLR, PQLR, RQLR, or RRLR.

41. The library of claim 38, wherein P₄P₃P₂P₁ comprises PRLR.

42. The library of claim 38, wherein P₄P₃P₂P₁ comprises PKLK, PKLR, or PRLK.

43. The library of claim 37, the putative hepsin substrates further comprising a fluorescence resonance energy transfer pair having a first member coupled to the one or more prime positions and a second member coupled to the one or more non-prime positions.

44. The library of claim 43, wherein the fluorescence resonance transfer energy pair comprises amino benzoic acid and nitro-tyrosine; 7-methoxy-3-carbamoyl-4-methylcoumarin and dinitrophenol, or 7-dimethylamino-3-carbamoyl-4-methylcoumarin and dabsyl.

45. A prodrug comprising:
an amino acid sequence and a cytotoxic moiety;
which amino acid sequence is selected from the group consisting of KRLR, KQLR, PRLR, PQLR, RQLR, RRLR, PKLK, PKLR, or PRLK.

46. The prodrug of claim 45, wherein the cytotoxic moiety comprises doxorubicin, daunorubicin, epirubicin, idarubicin, anthracycline, paclitaxel, mitomycin C, or phenylenediamine mustard.

47. A diagnostic compound comprising an amino acid sequence and a label moiety, the amino acid sequence comprising KRLR, KQLR, PQLR, RQLR, RRLR, PRLR, PKLK, PKLR, or PRLK.

48. The diagnostic compound of claim 47, wherein the label moiety comprises a chromaphore, a fluorophore, a coumarin moiety, a rhodamine moiety, or a fluorescence resonance transfer energy pair.

49. The diagnostic compound of claim 48, wherein the coumarin moiety comprises 7-amino-4-carbamoylcoumarin, 7-amino-3-carbamoyl-4-methylcoumarin, or 7-amino-4-methylcoumarin.

50. The diagnostic compound of claim 48, wherein the fluorescence resonance transfer energy pair comprises amino benzoic acid and nitro-tyrosine; 7-methoxy-3-carbamoyl-4-methylcoumarin and dinitrophenol, or 7-dimethylamino-3-carbamoyl-4-methylcoumarin and dabsyl.

51. The diagnostic compound of claim 47, the compound further comprising polyethylene glycol, a polysaccharide, or a saccharide.

52. A hepsin inhibitor comprising a hepsin recognition site, wherein the hepsin inhibitor comprises:
P₄P₃P₂P₁Z
wherein:
P₁ is arginine or lysine;
P₂ is valine, leucine, isoleucine, methionine, norleucine, arginine, histidine, lysine, asparagine, or threonine;
P₃ is arginine, lysine, histidine, glutamine, serine, or threonine;
P₄ is arginine, lysine, proline, valine, leucine, isoleucine, methionine, norleucine, alanine, glycine, tryptophan, phenylalanine, or tyrosine; and
Z comprises a transition state analog, a mechanism-based inhibitor, or an electron withdrawing group; and
wherein contacting hepsin with the hepsin inhibitor results in inactivation of the hepsin.

53. The hepsin inhibitor of claim 52, wherein P₁ comprises arginine, P₂ comprises leucine, P₃ comprises arginine, and P₄ comprises lysine.

54. The hepsin inhibitor of claim 52, wherein P₄ comprises acetyl-lysine.

55. The hepsin inhibitor of claim 52, wherein the transition state analog, mechanism-based moiety, or electron withdrawing moiety comprises a C-terminal aldehyde, a boronate, a phosphonate, an α-ketoamide, a chloro methyl ketone, a sulfonyl chloride, ethyl propenoate, vinyl amide, vinyl sulfone, vinyl sulfonamide.

56. A hepsin inhibitor comprising a compound having the chemical structure:

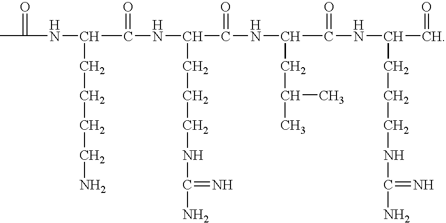

57. An expression vector for expression of a hepsin polypeptide in insect cells, the expression vector comprising the following operably linked components:
a promoter that is active in insect cells;
a polynucleotide that encodes a secretion signal polypeptide; and
a polynucleotide that encodes the hepsin polypeptide.

58. The expression vector of claim 57, wherein the hepsin polypeptide comprises a hepsin catalytic domain and prodomain.

59. The expression vector of claim 57, wherein the hepsin polypeptide lacks a transmembrane domain.

60. The expression vector of claim 57, wherein the secretion signal polypeptide is a non-hepsin secretion signal polypeptide.

61. The expression vector of claim 60, wherein the secretion signal polypeptide is a honeybee melittin secretion signal polypeptide.

62. The expression vector of claim 57, wherein the expression vector further comprises a polynucleotide that encodes a tag that facilitates purification of the hepsin polypeptide.

63. The expression vector of claim 62, wherein the tag is a polyhistidine tag.

64. A method of killing a cell, the method comprising: contacting the cell with a hepsin-cleavable molecule that comprises a hepsin cleavage site, wherein the hepsin-cleavable molecule comprises:
P₄P₃P₂P₁X
wherein the hepsin cleavage site is between P₁ and X; and
wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a cytotoxic moiety.

65. The method of claim 64, wherein the cytotoxic moiety comprises doxorubicin, daunorubicin, epirubicin, idarubicin, anthracycline, paclitaxel, camptothecin, mitomycin C, phenylenediamine mustard, or a bacterial toxin.

66. The method of claim 64, wherein the cell comprises a mammalian cell.

67. The method of claim 64, wherein contacting the cell with a hepsin-cleavable molecule is performed in vitro.

68. The method of claim 64, wherein contacting the cell with a hepsin-cleavable molecule comprises administering the hepsin cleavable molecule to the cell in vivo.

69. The method of claim 68, wherein the cell is a mammalian cell.

70. The method of claim 68, wherein the cell is a human cell.

71. A method of reducing a hepsin activity in a cell, the method comprising: contacting the cell with a hepsin inhibitor molecule that comprises a hepsin recognition site, wherein the hepsin inhibitor molecule comprises:
P₄P₃P₂P₁X
wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and
wherein X comprises a transition state analog, a mechanism-based inhibitor, or an electron withdrawing group.

72. The method of claim 69, wherein the cell is in cell culture.

73. The method of claim 69, wherein the cell is in a mammal.

74. The method of claim 69, wherein the cell is in a human.

75. The method of claim 69, wherein the hepsin inhibitor is applied to the cell in a pharmaceutically acceptable excipient.

76. A method of labeling a cell, the method comprising contacting the cell with a hepsin-cleavable molecule that comprises a hepsin cleavage site, wherein the hepsin-cleavable molecule comprises:
P₄P₃P₂P₁X
wherein the hepsin cleavage site is between P₁ and X; and
wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a label moiety.

77. The method of claim 76, wherein the label moiety comprises a coumarin moiety.

78. The method of claim 76, wherein the label moiety comprises a member of a donor-acceptor FRET pair.

79. The method of claim 76, wherein the cell comprises a prostate tissue cell.

80. A method of screening an individual for a hepsin activity or expression, the method comprising:
a) obtaining a cell or tissue sample from the individual;
b) contacting the cell or tissue sample with one or more hepsin-cleavable molecules that comprise a hepsin cleavage site, wherein the hepsin-cleavable molecule comprises:
P₄P₃P₂P₁X
wherein the hepsin cleavage site is between P₁ and X; and
wherein P₁ is arginine or lysine; P₂ is valine, leucine, isoleucine, methionine, norleucine, arginine, histidine, lysine, asparagine, or threonine; P₃ is arginine, lysine, histidine, glutamine, serine, or threonine; P₄ is arginine, lysine, proline, valine, leucine, isoleucine, methionine, norleucine, alanine, glycine, tryptophan, phenylalanine, or tyrosine; and X comprises a label moiety; and
c) detecting a release of the label moiety from the hepsin cleavable molecule, thereby screening the individual for the hepsin activity or expression.

81. The method of claim 80, further comprising comparing the release of the label moiety from the individual to a standard hepsin activity level.

82. The method of claim 80, wherein the hepsin activity is diagnostic of a disease.

83. The method of claim 80, wherein the hepsin activity is diagnostic of prostate cancer.

84. A method of obtaining a substrate profile for a modulator of hepsin activity, the method comprising:
(a) providing a library of putative hepsin substrates, each of which comprises a putative hepsin recognition site, wherein:
(i) the putative hepsin recognition site comprises one or more non-prime positions and one or more prime positions, each of which positions is occupied by a substrate moiety, wherein the prime and non-prime positions flank a putative hepsin cleavage site;
(ii) the substrate moieties that occupy one or more of the non-prime positions are preselected to allow cleavage of the substrate at the putative hepsin cleavage site by the hepsin; and
(iii) the substrate moieties that occupy one or more of the prime positions vary among different members of the library of hepsin substrates;
(b) incubating the library in the presence of the hepsin; and
(c) monitoring cleavage of the putative hepsin substrates by the hepsin, thereby providing the substrate profile for the hepsin.

85. The method of claim 84, wherein a fluorescence donor moiety and a fluorescence acceptor moiety are attached to the putative hepsin substrates on opposite sides of the putative hepsin cleavage site, and wherein monitoring the cleavage of the putative hepsin substrates comprises detecting a fluorescence resonance energy transfer.

86. The method of claim 84, wherein monitoring comprises detecting a shift in the excitation and/or emission maxima of the fluorescence acceptor moiety, which shift results from release of the fluorescence acceptor moiety from the putative hepsin substrate by the hepsin activity.

87. The method of claim 84, wherein the one or more non-prime positions comprises a tetrapeptide sequence.

88. The method of claim 85, wherein the tetrapeptide is selected from the group consisting of KRLR, KQLR, PQLR, RQLR, RRLR, PRLR, PKLK, PKLR and PRLK.

89. A method of screening a library of compounds for a modulator of hepsin activity, the method comprising:
(a) providing a first library comprising a plurality of putative hepsin substrates having a structure P₄P₃P₂P₁X, wherein P₁, P₂, P₃ and P₄ comprise substrate moieties at non-prime side positions and X comprises a label moiety coupled at a prime side substrate moiety position;
(b) analyzing the first library to identify substrate moieties at one or more non-prime positions that result in cleavage of the putative hepsin substrate between P₁ and X by a hepsin protease;
(c) constructing a second library comprising the identified substrate moieties, wherein constructing the second library comprises:
(i) coupling a first member of a fluorescence resonance energy transfer (FRET) pair to a substrate moiety on an N-terminal side of a putative hepsin cleavage site, wherein the substrate moiety comprises an identified substrate moiety from the first library;
(ii) coupling a second member of the FRET pair to a prime substrate moiety position on a C-terminal side of the putative hepsin cleavage site; and
(iii) linking the compounds of (i) and (ii) together to form members of the second library;
(d) incubating the second library with the hepsin protease; and
(e) monitoring fluorescence resonance energy transfer between the members of the FRET pair, to identify one or more optimal prime substrate moieties, thereby providing the substrate profile for the enzyme.

90. The method of claim 89, wherein the fluorescent resonance energy pair comprises amino benzoic acid and nitro-tyrosine; 7-methoxy-4-carbomoylmethylcoumarin and dinitrophenol-lysine, or 7-dimethylamino-4-carbomoylmethylcoumarin and Dabsyl-Lysine.

91. The method of claim 89, wherein the prime substrate moiety comprises a tetrapeptide.

92. The method of claim 89, wherein X further comprises the substrate moiety P₁′P₂′P₃′P₄′, wherein
P₁′ is attached to P₁ and is methionine, norleucine, leucine, isoleucine, valine, alanine, tyrosine, or threonine;
P₂′ is alanine, phenylalanine, tyrosine, threonine, or histidine;
P₃′ is arginine, lysine, histidine, glutamine, serine, threonine, tyrosine, tryptophan, glycine, leucine or methionine; and
P₄′ is attached to the label moiety and is aspartic acid, glycine, proline, valine, or methionine.
